(19) United States
(12) Patent Application Publication
(10) Pub. No.: US 2026/0174474 A1
(43) Pub. Date: Jun. 25, 2026

(54) Bone Harvesting Devices for Bone Implantation

(71) Applicant: Allumin8, Inc., Springfield, MO (US)

(72) Inventors: Alyssa Huffman, Springfield, MO (US); Matthew Shomper, Xenia, OH (US); Michael J. Millela, JR., Imperial Beach, CA (US)

(73) Assignee: Allumin8, Inc., Springfield, MO (US)

(21) Appl. No.: 19/126,753

(22) PCT Filed: Nov. 4, 2023
(86) PCT No.: PCT/US2023/078762

§ 371 Date: May 2, 2025

Related U.S. Application Data
(60) Provisional application No. 63/422,640, filed on Nov. 4, 2022.

(51) Int. Cl. A61B 17/70; A61B 17/86; A61F 2/44

(52) U.S. Cl. CPC A61B 17/7037; A61B 17/7032; A61B 17/7035; A61B 17/864; A61B 17/8685; A61F 2/4455

(57) ABSTRACT

Provided herein are medical devices and methods for treating bone fractures. The medical device comprises a body, a scaffold within the body, and a means for focusing bone growth throughout the body. The device is designed to minimize shear stresses on a distal tip and distribute micromotion to encourage bony ingrowth. The scaffold may include a triply periodic minimal surface (TPMS) with a cubic repeating pattern defining walls within the scaffold, and the TPMS may be helically wrapped around a central axis of the device. The device may also house biologic agents and accommodate an autologous product. In some embodiments, the medical device is a bone screw or a pedicle screw, designed to reduce common issues such as screw loosening, screw backout, and rod breakage. The method of treatment involves implanting the medical device into a patient's bone to aid fracture healing.

This application claims the benefit of priority of U.S. Provisional Patent Application Ser. No. 63/422,640 filed Nov. 4, 2023, the disclosure of which is incorporated by reference in its entirety for all purposes.

This disclosure relates to a scaffold material that resembles natural bone, medical devices made from this scaffold material, and related methods for bone implantation.

Spinal fusion is a commonly indicated procedure for managing fractures, instability, and common degenerative conditions, including low back pain. Fusion techniques use bone grafts and hardware, such as pedicle screws, to encourage two vertebral bodies to grow together. Prior lumbar spinal implant designs are smooth, threaded pedicle screws secured in vertebral bone with rods to hold the height and angulation of the correction until fusion is achieved.

Low back pain has been estimated to impact 60-80% of people globally. Between 1998 and 2008, annual lumbar fusion surgeries performed in the United States increased from 77,682 to 210,407. Unfortunately, the overall failure rate of lumbar spine surgery is high, around 10-46%.

In a review of spinal fusion surgeries within the PubMed database, 11,692 patients were extracted. There were 3,646 complications, the mean age at surgery was 53.3 years (range: 25-77 years), mean follow-up was 3.49 years (range: 6 weeks-9.7 years). Major perioperative complications occurred at a mean rate of 18.5%. Minor perioperative complications occurred at a mean rate of 15.7%. Long-term complications occurred at a mean rate of 20.5%.

Despite advances in technology and surgical techniques, these rates have not changed substantially over the years. For example, lumbar interbody fusion (LIF) technology has advanced because of computer navigation, augmented reality, minimally invasive surgical (MIS) approaches, disc arthroplasty, bone stimulation pedicle screws, and bone void filler options. Nevertheless, the number of patients developing failed back surgery syndrome (FBSS) has increased continually because of the increased patient population and the high failure rates. (FBSS is when the outcome of the lumbar spinal surgery does not meet the patient's and surgeon's pre-surgical expectations.)

The gold standard for lumbar interbody fusions consists of inserting a smooth threaded screw into each pedicle (two per vertebral level) and placing rods into the tulip heads of the pedicle screws to stabilize the construct until fusion is achieved. However, little technological advancement has been achieved in spinal stabilization systems since 1975, and these constructs do not address key long-term stability issues related to the quality of bone mineral density and patient health. Hardware used in lumbar fusions experiences significant forces that cause hardware breakage and loosening (the so-called “Windshield Wiper Effect”). Estimates for the frequency of screw loosening during spinal fusions vary significantly, but a recent report estimated a loosening rate of over 40%, with nearly 10% being a partial pullout. This loosening due to lack of fusion can put nerves or blood vessels at risk, often requiring hardware to be removed and surgery to be repeated.

Unfortunately, revision spine surgery also suffers from poor success rates. Secondary revision cases have a success rate of 30%, third surgeries are 15%, and a fourth surgical intervention is 5%. In addition, revision adult spinal deformity patients who endured two or more previous demonstrate more coronal and sagittal imbalance and worse functional status. Other potential complications for LIF are dural tears, neurological injuries, pseudoarthrosis, infection, and wound healing issues.

Another complication of lumbar spinal fusion is infection. Surgical site infections (SSI) represent a major healthcare challenge and account for approximately 8,000 yearly deaths.21 The combined estimated direct and indirect costs stemming from SSI are between $1 and $10 billion per year. Spinal instrumented surgeries are at greater risk for SSIs, resulting in higher rates of infection relative to other orthopedic surgeries. SSIs stemming from spinal surgeries are estimated to occur between 0.2% and 16.7% of the time. A recent meta-analysis suggests instrumented spinal surgeries result in SSI 4.4% of the time. Deep incisional and organ space SSIs account for 80% of these infections and are linked to increased morbidity, longer hospital stays, and greater medical expenses.

Moreover, bone mineral density decreases after medical devices are implanted, regardless of the material used. This loss contributes to common medical device failures, including screw loosening, screw backout, and rod breakage. Though many devices promote fusion in an interbody cage, none have been developed for scaffolding and increasing bone mineral density within the vertebral body. Also, the structure of cortical bone within the vertebrae differs from the bone in other parts of the human body.

These challenges relating to long-term stability, such as bone quality and functional ability to heal, have not been satisfied. None of the prior technologies have addressed the top two reasons for implant failure revision surgery: pedicle screw backout and rod breakage before the patient achieves fusion. Smooth, threaded pedicle screws and rods do not address the quality of bone mineral density and patient health.

### SUMMARY

The present disclosure provides a medical device that comprises a body, a scaffold located within the body, and a means for focusing bone growth that is disposed on the body. The design of the device is such that it minimizes shear stresses on a distal tip and spreads micromotion throughout the medical device, thereby encouraging bony ingrowth.

The present disclosure also provides a method of treating a bone fracture in a patient in need thereof, comprising implanting a medical device into a bone of the patient. The medical device may be any one of the devices disclosed herein.

Additional embodiments and features are set forth in part in the following description. They will become apparent to those skilled in the art upon examination of the specification or may be learned by the practice of the embodiments discussed herein. A further understanding of the nature and advantages of certain embodiments may be realized by reference to the remaining portions of the specification and the drawings, which form a part of this disclosure.

### BRIEF DESCRIPTION OF THE DRAWINGS

FIG. 1 is a top view of a diagram showing a pedicle screw implanted through a pedicle into the vertebral body of a vertebra.

FIG. 2 shows a side plan view of an embodiment of a pedicle screw 3D-printed in titanium.

FIG. 3 shows a top plan view of the pedicle screw of FIG. 2.

FIG. 4 shows a bottom plan view of the pedicle screw of FIG. 2.

FIG. 5 shows a side plan view of an embodiment of a pedicle screw 3D-printed with scaffold disclosed herein.

FIG. 6 shows a top plan view of the pedicle screw of FIG. 5.

FIG. 7 shows a bottom plan view of the pedicle screw of FIG. 5.

FIG. 8 shows a side plan view of another embodiment of a pedicle screw 3D-printed with scaffold disclosed herein.

FIG. 9 shows a top plan view of the pedicle screw of FIG. 8.

FIG. 10 shows a bottom plan view of the pedicle screw of FIG. 8.

FIG. 11 shows a side plan view of an embodiment of a porous pedicle screw.

FIG. 12 shows an exploded view of the porous pedicle screw of FIG. 11 with a tulip and pins.

FIG. 13 shows a top plan view of the cap of the porous pedicle screw of FIG. 11.

FIG. 14 shows a side plan view of the cap of the porous pedicle screw of FIG. 11.

FIG. 15 shows a perspective view of the saddle of the porous pedicle screw of FIG. 11.

FIG. 16 shows a perspective view of the tulip of FIG. 12.

FIG. 17 shows a screw with bone harvesting features located along the distal portion of the screw but not along the proximal portion of the screw.

FIG. 18 shows the proximal portion of the screw of FIG. 17 without bone harvesting features.

FIG. 19 shows the distal portion of the screw of FIG. 17 having bone harvesting features.

FIG. 20 shows cross-section A-A from the screw of FIG. 17.

FIG. 21 shows cross-section B-B from the screw of FIG. 17.

FIG. 22 illustrates a 2-dimensional slice through the Schwarz Diamond mathematical field.

FIG. 23 illustrates a 3-dimensional cross-section of the non-thickened surface from FIG. 22.

FIG. 24 shows the cylindrical remap after conversion to polar space of the non-thickened surface of FIG. 23.

FIG. 25 shows the remap of FIG. 24 without shear.

FIG. 26 shows the remaps of FIG. 25 with shear.

FIG. 27 shows the thickened surface of FIG. 26 using an absolute value operation.

FIG. 28 shows the sheet-like structure of the Schwarz Diamond lattice after thickening and subtraction.

FIG. 29 shows the thin-walled lattice field of FIG. 28 intersected with the 3D geometry space that defines where the lattice exists to define the scaffold.

FIG. 30 shows a perspective view of a porous pedicle screw presenting a diamond-structured lattice.

FIG. 31 shows the back view of the porous pedicle screw of FIG. 30.

FIG. 32 shows the front view of the porous pedicle screw of FIG. 30.

FIG. 33 shows a magnified inset of the front view of the porous pedicle screw of FIG. 30, highlighting the scaffold.

FIG. 34 shows the top view of the porous pedicle screw of FIG. 30.

FIG. 35 shows the bottom view of the porous pedicle screw of FIG. 30.

FIG. 36 shows a perspective view of another embodiment of a porous pedicle screw presenting a diamond-structured lattice comprising bone harvesting features at the distal tip and textured threads at the proximal end.

FIG. 37 shows the back view of the porous pedicle screw of FIG. 36.

FIG. 38 shows the front view of the porous pedicle screw of FIG. 36.

FIG. 39 shows a magnified inset of the front view of the porous pedicle screw of FIG. 36, highlighting the scaffold.

FIG. 40 shows the top view of the porous pedicle screw of FIG. 36.

FIG. 41 shows the bottom view of the porous pedicle screw of FIG. 36.

The disclosure will be readily understood by the following detailed description in conjunction with the accompanying drawings, wherein like reference numerals designate like structural elements. The drawings provide exemplary embodiments or aspects of the disclosure and do not limit the scope of the disclosure.

### DETAILED DESCRIPTION

The present disclosure provides a medical device that comprises a body, a scaffold located within the body, and a means for focusing bone growth that is disposed on the body. The design of the device is such that it minimizes shear stresses on a distal tip and spreads micromotion throughout the medical device, thereby encouraging bony ingrowth.

In certain embodiments, the means for focusing bone growth in the medical device comprises at least one trephine to harvest bone internally within the device.

In certain embodiments, the medical device, whether it includes the trephine or not, is configured in arcuate cross-section patterns that vary from a proximal end to a distal tip

In certain embodiments, the medical device is configured for placement into an internal cavity of a vertebral body. Upon coaxial rotation of the medical device, an autograft is harvested within the scaffold.

In certain embodiments, the medical device, regardless of its other features, comprises threads with a concave profile.

In certain embodiments, the scaffold within the medical device comprises a triply periodic minimal surface (TPMS) with a cubic repeating pattern defining walls within the scaffold.

In certain embodiments, the TPMS is a Schwartz Diamond helically wrapped around a central axis of the medical devices and into the 3D geometry space for the scaffold.

In certain embodiments, the TPMS is helically wrapped into a single helix. In certain embodiments, the TPMS is helically wrapped into a double helix. In certain embodiments, the TPMS is helically wrapped into a triple helix. In certain embodiments, the TPMS is helically wrapped into a quadruple helix. In certain embodiments, the medical device comprises three radial spokes per turn of the helix, whether the TPMS is helically wrapped into a single helix or not.

In certain embodiments, the cubic repeating pattern within the medical device is between about 1 mm and 3 mm in the X/Y/Z dimensions, such as between about 1.5 mm and 2 mm, or about 1.8 mm in the X/Y/Z dimensions. In certain embodiments, the helical wrap of the medical device is defined with a period three times the size of the cubic repeating pattern. In certain embodiments, the walls within the medical device are about 0.5 mm thick, regardless of other features.

In certain embodiments, the threads near the proximal ends of the medical device are textured with a topography resembling the topography of the scaffold.

In certain embodiments, the medical device is configured to house one or more biologic agents, regardless of its other features. In certain embodiments, the medical device further comprises at least one autologous product sprayed on or injected through the device.

In certain embodiments, the body of the medical device is a shaft. In certain embodiments, the medical device is a bone screw. In certain embodiments, the medical device has reduced one or more of screw loosening, screw backout, rod breakage, and lowered bone mineral density. In certain embodiments, the medical device comprises a fluted tip.

In certain embodiments, the medical device is a pedicle screw.

The present disclosure also provides a method of treating a bone fracture in a patient in need thereof, comprising implanting a medical device into a bone of the patient. The medical device may be any one of the devices disclosed herein.

### Scaffold

Surface curvature and Minkowski bone morphology curvature maps (a function that recovers a notion of distance on a linear space) demonstrate a significantly different porous matrix in trabecular bone within the vertebrae versus other areas of skeletal anatomy. A load of traditional smooth, threaded pedicle screws can be too high for vertebral bone, as bone mineral density lowers post-implantation.

Porous 3D-printed scaffolds promote osseointegration, fusion, and fixation within a bone. The open skeleton with a scaffold is similar to that of native bone. This similarity allows physicians to make patient-specific choices using other agents to promote bone formation and/or stabilize the device.

Triangular-shaped porosity sequences have been used in the prior art. Rounded, square/rectangular shapes and variegated patterns more closely align with native vertebral bone structure. In addition, the scaffold's structure reduces the likelihood of revision of the medical device from which it is made, for example, screw loosening, screw backout, rod breakage, and lower bone mineral density.

In certain embodiments, the disclosed scaffold and devices integrate orthopedic products with regenerative medicine to prevent the risks of delayed bone fusion to implanted devices.

In certain embodiments, the scaffold comprises one or more structural cues chosen from porosity, pore size, grain size, and surface topography. Porosity and pore size cue signal mechanical strength, cell settlement, and cell migrations. Grain size cures signal protein absorption, cell adhesion, cell proliferation, and cell adhesion. Surface topography cures signal-specific surface area, cell adhesion, and a material-tissue interface. Other scaffold features include pH and wall thickness. In certain embodiments, the one or more structural cues enhance at least one of multipotent mesenchymal stem cell (MSC) differentiation, osteoblast growth, extracellular matrix (ECM) deposition, and new bone formation. In certain embodiments, the new bone formation occurs after MSC differentiation, osteoblast growth, ECM deposition, or combinations thereof.

### Bone

Bones can generally be divided into cancellous bone and cortical bone. “Cancellous bone,” also called “trabecular bone” or “spongy bone,” is a light, porous bone enclosing numerous large spaces that give a honeycombed or spongy appearance. The bone matrix, or framework, is organized into a three-dimensional latticework of bony processes, called trabeculae, arranged along stress lines. The spaces between are often filled with marrow and blood vessels. In cross-sections, trabeculae of a cancellous bone can look like septa. But, they are topologically distinct in three dimensions, with trabeculae roughly rod or pillar-shaped and septa sheet-like.

Cancellous bone makes up about 20% of the human skeleton, providing structural support and flexibility without compact bone. It is found in most areas of bone not subject to great mechanical stress. It makes up much of the enlarged ends (epiphyses) of the long bones and is the major component of the ribs, the shoulder blades, the flat bones of the skull, and a variety of short, flat bones elsewhere in the skeleton.

Because of the increasing frequency of total joint replacements and their impact on bone remodeling, understanding the stress-related and adaptive process of trabecular bone has become a central concern for bone physiologists. To understand the role of trabecular bone in age-related bone structure and design for the bone-implant system, the mechanical properties of trabecular bone are studied as a function of the anatomic site, density, and age. So, mechanical factors, including modulus, uniaxial strength, and fatigue properties, are also studied.

High porosity makes trabecular bone compliant. Large variations in architecture lead to high heterogeneity. The modulus and strength vary inversely with porosity and highly depend on the porosity structure. Typically, the porosity percent of cancellous bone is between 75% and 95%. The density is between 0.2 and 0.8 g/cm³. Porosity can reduce the strength of the bone but also reduce its weight.

The porosity and its structure affect the strength of the material. Thus, the microstructure of trabecular bone is typically oriented. The “grain” of porosity is aligned where mechanical stiffness and strength are the greatest. Because of the microstructural directionality, the mechanical properties of trabecular bone are highly anisotropic. Young's modulus for trabecular bone, including vertebral bone, is between 800 and 14,000 Mpa. Its strength of failure is 1 to 100 MPa.

“Cortical bone” or “compact bone” is much denser than cancellous bone. It forms the hard exterior (cortex) of bones. The cortical bone gives bone its smooth, white, and solid appearance. It accounts for about 80% of the total bone mass of an adult human skeleton. Cancellous bone is usually surrounded by a shell of compact bone, which provides greater strength and rigidity. The open structure of cancellous bone enables it to dampen sudden stresses, as in load transmission through the joints. Varying proportions of space to bone are found in different bones according to the need for strength or flexibility. Cancellous bone also has a relatively high level of metabolic activity.

“Wolff's law” refers to the bone in a healthy person or animal adapting to the loads under which it is placed. For example, if loading on a particular bone increases, it will remodel itself over time to become stronger to resist that loading.

### Vertebrae

Each vertebra is an irregular bone with a complex structure composed of bone and some hyaline cartilage in the vertebrate spinal column. The proportions vary according to the segment of the backbone and vertebrate species.

The basic configuration of vertebrae varies. The large part is the body, and the central part is the centrum. The upper and lower surfaces of the vertebra body give attachment to the intervertebral discs. The posterior part forms a vertebral arch in eleven parts, consisting of two pedicles, two laminae, and seven processes. The laminae give attachment to the ligamenta flava (ligaments of the spine). There are vertebral notches formed from the shape of the pedicles, which form the intervertebral foramina when the vertebrae articulate. These foramina are the entry and exit conduits for the spinal nerves. The body of the vertebra and the vertebral arch form the vertebral foramen, the larger, central opening that accommodates the spinal canal, which encloses and protects the spinal cord.

Pedicles and laminae form the vertebral arch. Two pedicles extend from the sides of the vertebral body to join the body to the arch. The pedicles are short thick processes that extend, one from each side, posteriorly, from the junctions of the posterolateral surfaces of the centrum, on its upper surface. From each pedicle, a broad plate, called a “lamina,” projects backward and medialward to join and complete the vertebral arch and form the posterior border of the vertebral foramen, which completes the triangle of the vertebral foramen. The upper surfaces of the laminae are rough to give attachment to the ligamenta flava. These ligaments connect the laminae of the adjacent vertebra along the length of the spine from the level of the second cervical vertebra. Above and below the pedicles are shallow depressions called vertebral notches (superior and inferior). When the vertebrae articulate, the notches align with those on adjacent vertebrae, forming the intervertebral foramina openings. The foramina allow spinal nerves and associated blood vessels to enter and exit each vertebra. The articulating vertebrae provide a strong pillar of support for the body.

### Devices

The present disclosure provides a device formed from a scaffold disclosed herein. In certain embodiments, the device is cannulated and fenestrated with the scaffold. In certain embodiments, the device comprises a threaded distal region, an optionally threaded central region, and an optionally threaded proximal region, depending on the compressive forces.

In certain embodiments, the device is chosen from pedicle screw, cannulated pedicle screw, fenestrated pedicle screw, large-headed screw, small-headed screw, headless screw, trauma hip fracture device, glenoid cage, screws for a glenoid cage, trauma plate, tibial stem, femoral stem, hammertoe implant, nail fusion system, Charcot foot deformity correction, radial head fracture device, high tibial osteotomy, deformity correction, corpectomy cage, oncological correction, anchor, dental implant, maxillofacial implant, and sports medicine anchor.

In certain embodiments, the device is chosen from a hip fracture system, reverse total shoulder, dental implant, upper extremity hardware, lower extremity hardware, total joint replacement implants, total joint revision implant, spine fusion, spine arthroplasty, regenerative therapy, cartilage implant, maxillofacial hardware, and cardiac implant.

In some embodiments, the screw is configured with features that facilitate bone to grow through the structure of the screw from opposing sides allowing the bone to connect through the screw. In some embodiments, the structure is narrow, such as through the screw thread, thereby permitting rapid through growth. In some embodiments, the structure is deeper, such as through the minor diameter, thus bonding stronger. In some embodiments, the feature is a void in the screw or porous or structured to promote bone growth. In some embodiments, the structure collects autografts within the channels inside the device. In some embodiments, the feature is impregnated with one or more polymers.

In some embodiments, the device is configured to enhance the stabilization and fixation of bone screws within the bone and improve bone mineral density. In some embodiments, the device includes a spinal implant configured for engagement with cortical bone and cancellous bone within the vertebra. In some embodiments, the device is configured to resist and/or prevent toggling on a bone screw when the bone screw is engaged with dense cortical bone and a less dense cancellous bone resulting from a load on the bone screw. In some embodiments, the device is configured to resist and/or prevent loosening of the bone screw from the cortical bone and, in some instances, pull it out from the vertebra. In some embodiments, the device is configured to facilitate bone through-growth to improve bone attachment to the bone screw. In some embodiments, the bone screw is anchored in the bone, thereby reducing pullout. In some embodiments, the bone screw is designed to spread micromotion and reduce shearing to strengthen bone mineral density.

In some embodiments, the device includes a bone screw having bone through-growth through the shaft of the screw to reduce toggle and potential failure of the screw. In some embodiments, the bone screw includes features that allow the bone to grow through the structure of the bone screw from opposing sides allowing bone to connect through those bone screw structures. In some embodiments, the bone screw includes features that may be narrow, such as through the bone screw thread, which would allow for rapid through-growth. In some embodiments, the bone screw includes features that may be deeper, such as through the minor diameter, which would provide a larger volume of bone through-growth. In some embodiments, the bone screw includes features that may be a void or cavity through opposite sides of the bone screw and/or a void or cavity that enters and exits from the same or adjoining surfaces. In some embodiments, the void or cavity may contain a scaffold for the bone to attach or a porous structure on the surface of the void.

In some embodiments, the bone screw includes features or structures that may be disposed along a shaft portion of the bone screw. In some embodiments, the bone screw includes features or structures that may be disposed continuously along a surface of the bone screw, such as, for example, along a distal end. In some embodiments, the bone screw includes features or structures that may be disposed discontinuously along a portion of the bone screw. In some embodiments, the bone screw includes features or structures that may include a scaffold or polymers.

In some embodiments, the device comprises a spinal implant having a hybrid configuration that combines a manufacturing method, such as, for example, one or more prior manufacturing features and materials, and a manufacturing method, such as, for example, one or more additive manufacturing features and materials. In some embodiments, additive manufacturing includes 3-D printing. In some embodiments, additive manufacturing includes fused deposition modeling, selective laser sintering, direct metal laser sintering, selective laser melting, electron beam melting, layered object manufacturing, and stereolithography. In some embodiments, additive manufacturing comprises one or more chosen from rapid prototyping, desktop, direct, digital, instant, and on-demand manufacturing. In some embodiments, the device comprises a spinal implant manufactured by a fully additive process and grown or otherwise printed.

In certain embodiments, the devices comprises one or more chosen from demineralized bone matrix (DBM), pre-packed DBM, pre-packed synthetic DBM, unpacked DBM, and magnesium-infused titanium.

In some embodiments, the device comprises a spinal implant, such as, for example, a bone screw manufactured by combining traditional manufacturing methods and additive manufacturing methods. In some embodiments, the bone screw is manufactured by applying additive manufacturing material where the bone screw can benefit from the materials and properties of additive manufacturing. In some embodiments, traditional materials are used where the benefits, such as physical properties and cost, are superior to those resulting from additive manufacturing features and materials.

In some embodiments, the device treats a spinal disorder chosen from degenerative disc disease, disc herniation, osteoporosis, spondylolisthesis, stenosis, scoliosis, other curvature abnormalities, kyphosis, tumor, and fractures.

“Treating” or “treatment” of a disease or condition refers to performing a procedure that may include administering one or more drugs to a patient, employing implantable devices, and/or employing instruments that treat the disease, such as microdiscectomy instruments to remove portions bulging or herniated discs, and/or bone spurs, to alleviate signs or symptoms of the disease or condition. Treating or treatment does not require complete alleviation of signs or symptoms, does not require a cure, and specifically includes procedures that have a marginal effect on the patient. For example, treatment can include inhibiting the disease, e.g., arresting its development, or relieving the disease, e.g., causing regression.

“Prevention” refers to alleviation before signs or symptoms of a disease or condition appear. Thus, prevention includes preventing the disease from occurring in a patient who may be predisposed to the disease but has not yet been diagnosed as having it.

“Tissue” includes soft tissue, ligaments, tendons, cartilage, and/or bone. In certain embodiments, the tissue is cancellous bone, cortical bone, or corticocancellous bone.

In some embodiments, devices are used with other osteal and bone-related applications, including diagnostics and therapeutics. In some embodiments, devices are alternatively employed in surgical treatment with a patient in a prone or supine position and/or employ various surgical approaches to the spine, including anterior, posterior, posterior mid-line, lateral, posterolateral, and/or anterolateral approaches, and in other body regions such as maxillofacial and extremities. The devices may also be alternatively employed with procedures for treating the lumbar, cervical, thoracic, sacral, and pelvic regions of a spinal column. The devices may also be used on animals, bone models, and other non-living substrates, for example, in training, testing, and demonstration.

In certain embodiments, the device is a custom medical device. In certain embodiments, the device is adapted for sports medicine.

In certain embodiments, the device is temperature-sensing. In certain embodiments, the device is pH-balancing.

In certain embodiments, the devices are fabricated having a porosity with a porogen that is spheroidal, cuboidal, rectangular, elongated, tubular, fibrous, disc-shaped, platelet-shaped, polygonal, or a mixture thereof. In some embodiments, the porosity is based on a plurality of macropores, micropores, nanopores structures, and/or a combination thereof.

In certain embodiments, the device is fabricated from biologically acceptable materials suitable for medical applications, including metals, synthetic polymers, ceramics, bone material, and composites thereof. In certain embodiments, the device comprises one or more chosen from a metal, ceramic, rubber, hydrogel, rigid polymer, fabric, bone material, and composites thereof.

In certain embodiments, the device comprises a metal chosen from stainless steel alloys, aluminum, commercially pure titanium, titanium alloys, Grade 5 titanium, superelastic titanium alloys, magnesium-infused titanium, cobalt-chrome alloys, superelastic metallic alloys such as nitinol, super elastoplastic metals such as Gum Metal®. In certain embodiments, the device comprises a ceramic and composites thereof, such as calcium phosphate (e.g., Skelite™). In certain embodiments, the device comprises a rubber chosen from polyaryletherketone (PAEK), polyetheretherketone (PEEK), polyetherketoneketone (PEKK), polyetherketone (PEK), carbon-PEEK composites, PEEK-BaSO₄ rubber, polyethylene terephthalate (PET), silicone, polyurethane, silicone-polyurethane copolymer, and polyolefin rubber. In certain embodiments, the device comprises a hydrogel. In certain embodiments, the device comprises fabric. In certain embodiments, the device comprises a rigid polymer chosen from polyphenylene, polyimide, polyetherimide, polyethylene, and epoxy. In certain embodiments, the device comprises bone material chosen from autograft, allograft, xenograft, or transgenic cortical and/or corticocancellous bone. In certain embodiments, the device comprises tissue growth or differentiation factors. In certain embodiments, the device comprises resorbable materials, such as composites of metals and calcium-based ceramics, composites of PEEK and calcium-based ceramics, composites of PEEK with resorbable polymers, totally resorbable materials, such as calcium-based ceramics, for example, calcium phosphate, tri-calcium phosphate (TCP), hydroxyapatite (HA)-TCP, calcium sulfate, or other resorbable polymers, such as polyketide, polyglycolide, polytyrosine carbonate, polycaprolactone, and other combinations.

In certain embodiments, the device comprises a rubber chosen from polyaryletherketone (PAEK), polyetheretherketone (PEEK), polyetherketoneketone (PEKK), polyetherketone (PEK), carbon-PEEK composites, PEEK-BaSO₄ rubber, polyethylene terephthalate (PET), silicone, polyurethane, silicone-polyurethane copolymer, polyolefin rubber, synthetic collagen, and collagen matrix. In certain embodiments, the device comprises synthetic collagen. In certain embodiments, the device comprises collagen matrix.

In certain embodiments, the device comprises magnesium, vitamins, and minerals. “Vitamin” refers to an organic molecule (or a set of molecules closely related chemically, i.e. vitamers) that is an essential micronutrient that an organism needs in small quantities for the proper functioning of its metabolism. Some sources list fourteen vitamins, by including choline, but major health organizations typically list thirteen: vitamin A (as all-trans-retinol, all-trans-retinyl-esters, as well as all-trans-beta-carotene and other provitamin A carotenoids), vitamin B₁ (thiamine), vitamin B₂ (riboflavin), vitamin B₃ (niacin), vitamin B₅ (pantothenic acid), vitamin B₆ (pyridoxine), vitamin B₇ (biotin), vitamin B₉ (folic acid or folate), vitamin B₁₂ (cobalamins), vitamin C (ascorbic acid), vitamin D (calciferols), vitamin E (tocopherols and tocotrienols), and vitamin K (phylloquinone and menaquinones). In the context of nutrition, a “mineral” refers to a chemical element required as an essential nutrient by organisms to perform functions necessary for life, including potassium, chlorine, sodium, calcium, phosphorous, magnesium, iron, zinc, manganese, copper, iodine, chromium, molybdenum, selenium, and cobalt.

In certain embodiments, the device comprises a metal chosen from iron, stainless steel alloys, aluminum, commercially pure titanium, titanium alloys, Grade 5 titanium, superelastic titanium alloys, magnesium-infused titanium, cobalt-chrome alloys, superelastic metallic alloys such as nitinol, super elastoplastic metals such as Gum Metal®. In certain embodiments, the device comprises titanium. In certain embodiments, the device comprises iron.

In certain embodiments, the device is manufactured or 3D-printed from materials such as titanium, titanium alloy, cobalt chrome, carbon fiber, magnesium infused titanium, iron, or stainless steel. In certain embodiments, the device is manufactured from a shape memory alloy or shape memory polymer, allowing the device to conform to an anatomical shape of the patient's body.

In certain embodiments, the device comprises magnesium-infused titanium. In certain embodiments, the device comprises an angiotensin receptor blocker coating. In certain embodiments, the device comprises a type-1 cartilage collagen coating. In certain embodiments, the device is infused with an antibiotic.

In certain embodiments, the device is employed to treat an affected section of vertebrae. A medical practitioner obtains access to a surgical site, including the vertebrae, in any appropriate manner, such as through incision and retraction of tissues. In certain embodiments, the device comprises a bone screw to augment a surgical treatment. In certain embodiments, the device can be pre-assembled for delivery to a surgical site or assembled in situ. In certain embodiments, the device is entirely or partially revised, removed, or replaced.

In certain embodiments, the device is used with surgical methods or techniques, including, but not limited to, open surgery, mini-open surgery, minimally invasive surgery (MIS), and percutaneous surgical implantation, whereby the vertebra is accessed through a mini-incision or a sleeve provides a protected passageway to the area. Once access to the surgical site is obtained, a surgical treatment, such as a corpectomy or discectomy, can be performed to treat a disease or disorder.

In certain embodiments, the surface of the devices comprises a non-solid configuration, such as a lattice. In some embodiments, the non-solid configuration comprises a porous structure or a trabecular configuration.

In various embodiments, the non-solid configuration is configured to provide one or a plurality of pathways to aid bone growth within and through from one surface to an opposite surface of the device. In some embodiments, the lattice comprises one or more portions, layers, or substrates. In some embodiments, one or more portions, layers, or substrates of the lattice are disposed side by side, offset, staggered, stepped, tapered, end to end, spaced apart, in series, or parallel. In some embodiments, the lattice defines a thickness, which may be uniform, undulating, tapered, increasing, decreasing, variable, offset, stepped, arcuate, angled, and/or staggered. In some embodiments, one or more lattice layers are disposed in a side-by-side, parallel orientation within a wall. In certain embodiments, the lattice comprises one or more layers of a material matrix.

In some embodiments, the lattice comprises a plurality of nodes and openings disposed in rows and columns or randomly. In some embodiments, the plurality of nodes and openings are disposed in series. In some embodiments, the plurality of nodes and openings are disposed in parallel.

In some embodiments, the lattice forms a rasp-like configuration. In some embodiments, the lattice is configured to engage tissue. In certain embodiments, the engagement of the lattice is to cut, shave, shear, incise or disrupt the tissue. In some embodiments, the lattice comprises a configuration chosen from cylindrical, round, oval, oblong, triangular, polygonal having planar or arcuate side portions, irregular, uniform, non-uniform, consistent, variable, horseshoe shape, U-shape, or kidney bean shape. In some embodiments, the lattice is rough, textured, porous, semi-porous, dimpled, knurled, toothed, grooved, or polished, for example, to engage and cut the tissue. In some embodiments, the lattice forms a tunnel configured to guide, drive, or direct the cut tissue into an opening, such as fusing the device to the tissue.

### Screw

In certain embodiments, the device is a screw. In some embodiments, the screw is chosen from a posted screw, a pedicle screw, a bolt, a bone screw for a lateral plate, an interbody screw, a uniaxial screw, a fixed angle screw, a multi-axial screw, a side-loading screw, a sagittal adjusting screw, a transverse sagittal adjusting screw, an awl tip, a dual rod multi-axial screw, midline lumbar fusion screw, and/or a sacral bone screw.

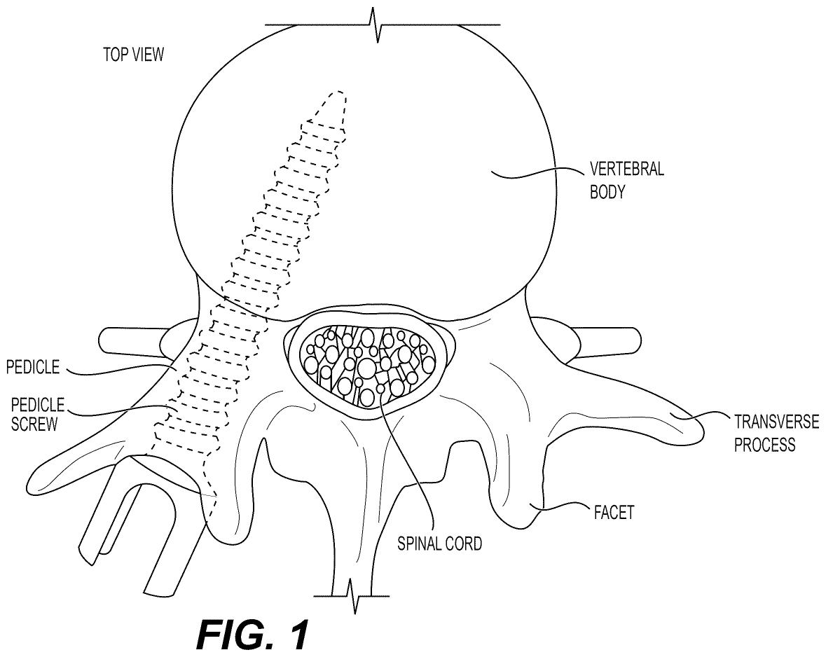

In certain embodiments, the device is a bone screw. In certain embodiments, the device is a pedicle screw. Referring to FIG. 1, a pedicle screw is shown implanted through a pedicle into the vertebral body of a vertebra. In certain embodiments, the pedicle screw has a cage with polymers held within the cannulated and/or fenestrated portion of the screw.

In certain embodiments, the internal core of the screw is a trephine to collect and harvest autograft upon and/or during insertion of the screw.

In certain embodiments, post-implantation options prevent revision surgery through polymer injection through the screw.

In certain embodiments, the pedicle screw does not exhibit screw loosening, screw backout, rod breakage, or lowered bone mineral density.

In certain embodiments, the pedicle screw has reduced one or more screw loosening, screw backout, rod breakage, and lowered bone mineral density.

The disclosed screws focus on bone growth throughout the shaft to minimize shear stresses on the distal tip and spread micromotion evenly throughout the screw to encourage bony ingrowth.

In certain embodiments, the scaffold of pedicle screw provides options for simple to complex bone mineral densities and immunocompromised patients. In certain embodiments, the scaffold is impregnated with one or more biologics, antibiotics, demineralized bone matrices, nanotechnology, or regenerative medicine therapies.

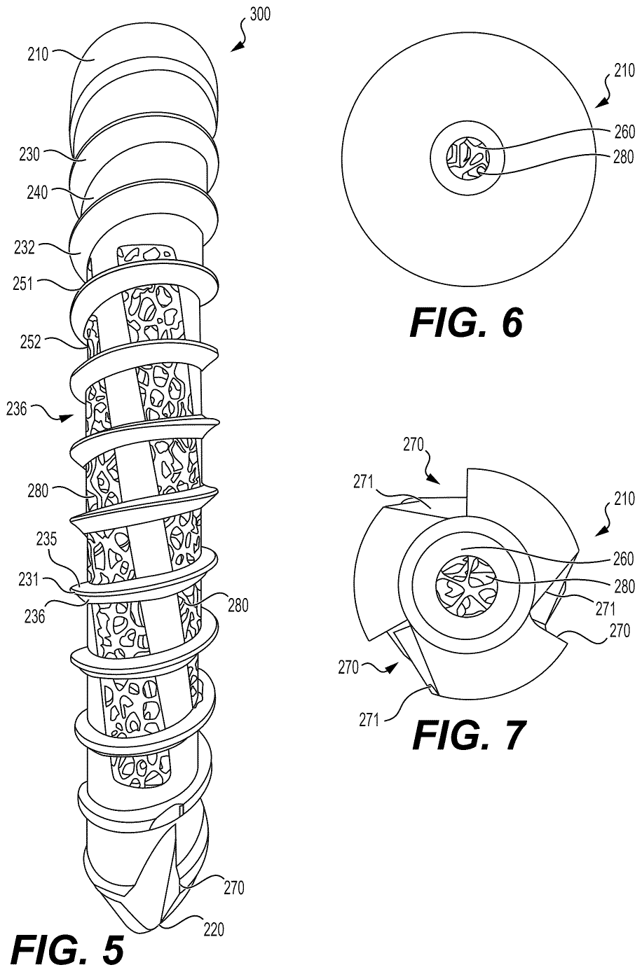

Referring to FIGS. 5-16, the structure of the pedicle screw 300,400,500 is specifically designed to help bone in-growth through the pedicle screw 300,400,500 by using the scaffold 280, similar to native trabecular bone in the vertebral body. In combination with the thread 230 and the scaffold 280, the core 260 aids autograft harvesting during the insertion process to push autograft into built-in channels within the core 260 of the pedicle screw 300,400,500. The walls surrounding the pores harvest the autograft and act as trephines. This structure also aided the pedicle screw's 300,400,500 structural integrity, resisted bone mineral density loss, and reduced micromotion.

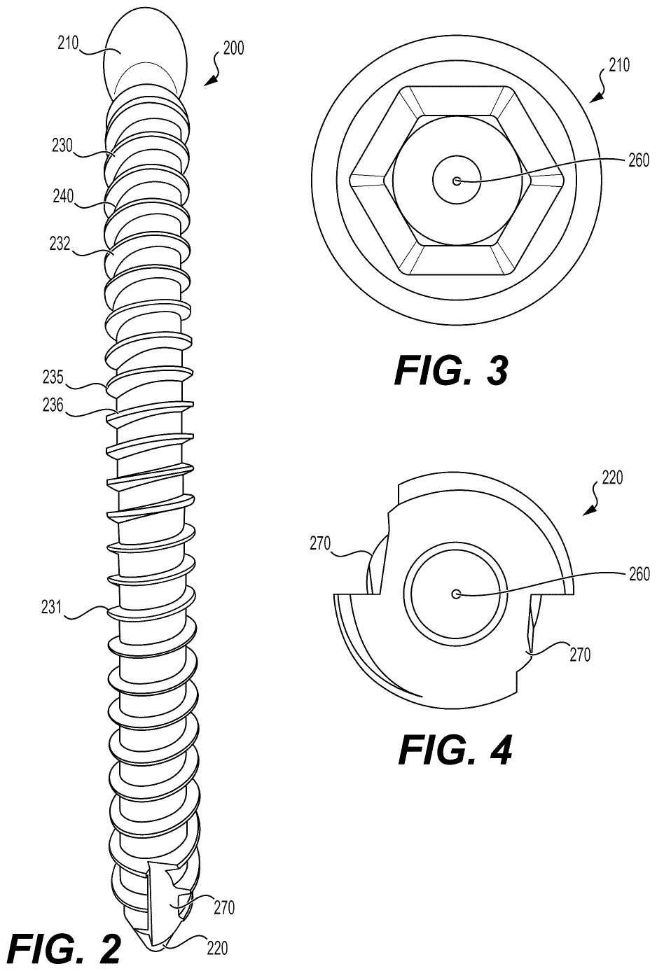

Referring to FIGS. 2-16, the pedicle screws 200,300,400,500 disclosed herein overcome the many failures of the prior art pedicle screws. In certain embodiments, the pedicle screw lacks a windshield wiper effect. In certain embodiments, the pedicle screw resists backout. In certain embodiments, the pedicle screw does not exhibit excessive micromotion. In certain embodiments, the pedicle screw has a low frequency of low-virulent microorganisms detected by sonication, for example, due to individual screw sterilization and packaging. In certain embodiments, the head and shaft of the pedicle screw resist failure. In certain embodiments, the pedicle screw is adapted for each type of bone quality. In certain embodiments, the pedicle screw has adequate thread depth. In certain embodiments, the pedicle screw withstands insertion torque, particularly at the head-to-screw coupling. In certain embodiments, the fatigue lifespan of the pedicle screw does not decrease when the screw is fully inserted. In certain embodiments, the pedicle screw has good instrumentation. In certain embodiments, the pedicle screw achieves angulation for rod acceptance. In certain embodiments, the pedicle screw does not have cyclic loading based on physiological conditions during walking. In certain embodiments, the pedicle screw does not fail in long-segment posterior cervical fusion, not requiring concomitant C6 or T1 buttress pedicles. In certain embodiments, the pedicle screw distributes stress. In certain embodiments, the pedicle screw does not immunocompromise the patient. In certain embodiments, the pedicle screw does not comprise PEEK. In certain embodiments, the pedicle screw does not have tulip or locking cap stresses.

In some embodiments, the distal tip 220 of the pedicle screw 200,300,400,500 has a surface configuration chosen from angled, irregular, uniform, non-uniform, offset, staggered, tapered, arcuate, undulating, mesh, porous, semi-porous, dimpled, pointed, textured, or combinations thereof. In some embodiments, the distal tip 220 includes a nail configuration, barbs, expanding elements, raised elements, ribs, and/or spikes to provide a fabrication platform for forming a portion thereon via additive manufacturing. In some embodiments, the distal tip 220 has a cross-section configuration chosen from oval, oblong triangular, square, polygonal, irregular, uniform, non-uniform, offset, staggered, tapered, or combinations thereof.

In certain embodiments, the pedicle screw 200,300,400,500 comprises a thread 230 that extends between the proximal end 210 and distal tip 220. In certain embodiments, the thread 230 comprises an external thread form. In certain embodiments, the thread form comprises a leading edge 231 having a leading surface 235 and a trailing edge 232 having a trailing surface 236. The leading surface 235 defines a first opening 251. The trailing surface 236 defines a second opening 252. In some embodiments, the first and second openings 251,252 are axially aligned. In some embodiments, the first and second openings 251,252 are disposed circumferentially about the thread form.

In some embodiments, the leading surface 235 and/or the trailing surface 236 comprises at least one tissue gathering member. In some embodiments, the tissue gathering member comprises a cutting edge. In some embodiments, the cutting edge is configured to be rasp-like. In some embodiments, the cutting edge is configured to engage tissue, for example, to cut, shave, shear, incise, or disrupt the tissue. In some embodiments, the cutting edge is configured to be cylindrical, round, oval, oblong, triangular, polygonal, having planar or arcuate side portions, irregular, uniform, non-uniform, consistent, variable, horseshoe shape, U-shape, or kidney bean shape. In some embodiments, the cutting edge is rough, textured, porous, semi-porous, dimpled, knurled, toothed, grooved, or polished to engage and cut the tissue. In some embodiments, the cutting edge forms a tunnel configured to guide, drive, or direct the cut tissue into the void, such as fusing the screw with the tissue.

For example, manipulating the pedicle screw 200,300,400,500 by rotation or translation causes the cutting edge 271 of the screw to cut and guide the tissue or bone into the core 260, thereby promoting bone growth and fusion to the pedicle screw 200,300,400,500. In some embodiments, the tissue is embedded into the core 260 to promote bone growth and fusion to the pedicle screw 200,300,400,500. In some embodiments, the lattice is disposed within the core 260 to form a scaffold 280 for bone growth.

In some embodiments, the thread 230 is configured as fine, closely spaced, or shallow to engage with the tissue. In some embodiments, the thread 230 comprises an increased pitch and an equal lead between thread turns. In some embodiments, the thread 230 comprises a smaller pitch or more thread turns per the axial distance to fixate stronger fixation with the tissue or resist loosening from the tissue. In some embodiments, the thread 230 is configured to be continuous along a portion. In some embodiments, the thread 230 is configured to be intermittent, staggered, or discontinuous. In certain embodiments, the thread 230 comprises a single thread turn.

In certain embodiments, the thread comprises a plurality of discrete threads. In certain embodiments, the thread has a concave profile.

In some embodiments, the thread 230 comprises a penetrating element, for example, chosen from a nail configuration, barb, expanding element, raised elements, rib, or spike. In some embodiments, the thread 230 is configured as self-tapping or intermittent at the distal tip 220. In some embodiments, the distal tip 220 is rounded. In some embodiments, the distal tip 220 is self-drilling. In some embodiments, the distal tip 220 comprises a solid outer surface.

In certain embodiments, the screw is a 3D-printed porous pedicle screw. Its porosity mimics native vertebral bone to attach and keep stem cells, growth factors, and other proteins within the structure of the pedicle screw and encourage bone growth through the screw, stabilizing the overall construct. During insertion into vertebral bone, the built-in trephines collect autograft and regenerative cells within the porous matrix. The disclosed topography attracts bone-forming stem cells within and around the device, reducing overall construct macromotion. In certain embodiments, this device enables surgeons to meet patient-specific needs, such as, but not limited to, spraying/injecting regenerative products to stimulate the bone-forming osteogenic cascade, proactively injecting the screw scaffold with antibiotics for diabetic-prone infections, and the option to inject bone cement to further stabilize the construct in severely osteoporotic bone.

In certain embodiments, the screw reduces revision rates, improves bone mineral density, and/or addresses patient-specific needs during spine fusions. In certain embodiments, bone mineral density improves, constructs are stabilized, and the likelihood of revision is reduced.

In certain embodiments, the screw is a 3D-printed titanium porous pedicle screw with a porous pattern throughout the screw similar to native bone. Without wishing to be bound by theory, the function of the porous pattern is to attach to the surrounding bone, keeping osteogenic stem cells in place and collecting autograft bone within its porous structure. An advantage of the porous structure is the ability to inject polymers and regenerative therapies through the screw. In certain embodiments, stem cell therapies are injected through the screw implant. In such embodiments, failure likelihood is reduced.

In certain embodiments, the surgeon can inject or spray the screw with autologous concentrated stem cells. Without wishing to be bound by theory, as the screw turns during insertion into the vertebrae, the screw's pores collect an autograft/stem cell mixture internally using its built-in trephines. The osteogenic stem cells then bind with the concentrated blood stem cells and signal the process of mutation and replication, forming more osteogenic cells within the screw, followed by a healing cascade of bone directed within and around the screw. In these embodiments, the combination of (a) osteoconductive (bone grows on the surface), (b) osteoinductive (recruiter of cells for bone healing), and (c) osteogenic (development and formation of bone) healing cascade of the stem cells improve bone mineral density and support superior bone integration and pullout strength.

In certain embodiments, the patient is diabetic and prone to infection. In these embodiments, the surgeon can inject a mixture comprising a calcium sulfate product and antibiotics through the screw before or after insertion or on the screw within the pedicle to provide antibiotic delivery in the area. In certain embodiments, the antibiotics are delivered for between two and six weeks. As such, the likelihood of revision due to infection is reduced.

### Manufacturing

The devices disclosed herein can be manufactured using various methods. In some embodiments, manufacturing comprises machining, such as subtractive, deformative, or transformative manufacturing. In some embodiments, manufacturing includes cutting, grinding, rolling, forming, molding, casting, forging, extruding, whirling, grinding, cold working, or combinations thereof. In some embodiments, manufacturing includes a portion of the device formed by a medical machining process. In some embodiments, machining uses computer numerical control (CNC) high-speed milling machines, Swiss machining devices, CNC turning with living tooling, wire EDM 4th axis, and combinations thereof. In some embodiments, the manufacturing for fabricating a portion of the devices includes a finishing process, such as laser marking, tumble blasting, bead blasting, micro blasting, powder blasting, or combinations thereof.

In certain embodiments, the device is fabricated per instructions from a computer and processor based on the digital rendering and/or data of a selected configuration via additive manufacturing.

In some embodiments, additive manufacturing comprises 3-D printing. In some embodiments, additive manufacturing is chosen from fused deposition modeling, selective laser sintering, direct metal laser sintering, selective laser melting, electron beam melting, layered object manufacturing, stereolithography, and combinations thereof. In some embodiments, additive manufacturing comprises rapid prototyping, desktop manufacturing, direct manufacturing, direct digital manufacturing, digital fabrication, instant manufacturing, on-demand manufacturing, or combinations thereof.

In some embodiments, a portion of the device is manufactured by additive manufacturing and then mechanically attached to a surface of the device, for example, by welding, threading, adhesives, or staking.

In one embodiment, the device is configured based on imaging from patient anatomy. Suitable imaging techniques include, but are not limited to, X-ray, fluoroscopy, computed tomography (CT), magnetic resonance imaging (MRI), surgical navigation, bone density (DEXA), or acquirable 2-D or 3-D images of patient anatomy. Selected configuration parameters for the device are collected, calculated, or determined. Examples of configuration parameters include, but are not limited to, patient anatomy imaging, surgical treatment, historical patient data, statistical data, treatment algorithms, implant material, implant dimensions, porosity, and manufacturing method. In some embodiments, the configuration parameters comprise implant material and device porosity based on patient anatomy and surgical treatment. In some embodiments, porosity is selected. In some embodiments, the configuration parameter of the device is patient specific. In some embodiments, the configuration parameter of the device is based on a generic configuration and is not patient specific.

For example, a digital rendering or data of a device is generated for display from a graphical user interface or storage on a database attached to a computer and a processor. In some embodiments, the computer display via a monitor saves, digitally manipulates, or prints a hard copy of the digital rendering or data. In some embodiments, the device is designed virtually with a CAD/CAM program on a computer display. In some embodiments, the processor executes code stored in a computer-readable memory medium to execute one or more computer instructions, for example, transmitting instructions to an additive manufacturing device. In some embodiments, the database or computer-readable medium comprises RAM, ROM, EPROM, magnetic, optical, digital, electromagnetic, flash drive, semiconductor technology, or combinations thereof. In some embodiments, the processor instructs motors to control the movement and rotation of device components.

### Regenerative Medicine

“Regenerative medicine” refers to a branch of translational research in tissue engineering and molecular biology, which deals with replacing, engineering, or regenerating human cells, tissues, or organs to restore or establish normal function. This field holds the promise of engineering damaged tissues and organs by stimulating the repair mechanisms within the patient's body to functionally heal previously irreparable tissues or organs. For example, during bone regeneration, new bone formation is primarily affected by physicochemical cues in the surrounding microenvironment. Tissue cells reside in a complex scaffold physiological microenvironment.

In certain embodiments, regenerative medicine is incorporated with the scaffolds or devices disclosed herein. Autogenous graft incorporation occurs in five stages: inflammation, vascularization, osteoinduction, osteoconduction, and remodeling.

Inflammation lasts for about 7 to 14 days. Initial insult to the local blood supply and decortications results in hematoma around the bone graft, in which inflammatory cells invade. The fibroblast-like cells in the inflammatory tissue transform into the fibrovascular stroma. Perioperative anti-inflammatory medications decrease fusion rates because of the inflammatory process.

Vascular buds appear in the fibrovascular stroma, resembling scar tissue formation during vascularization. Primary membranous bone forms near the decorticated bone. Next, minimal cartilage and endochondral ossification occur.

During osteoinduction at weeks 4 to 5, reparation comprises increased vascularization, necrotic tissue resorption, osteoblasts, and chondroblasts differentiation. In particular, stem cells differentiate into osteoblasts. New bone extends towards the central zone of the fusion mass. The cortical portion of the graft continues to resorb.

Osteoconduction is characterized by ingrowth into the host bone and creeping substitution. Osteoblasts create new bone while osteoclasts simultaneously resorb graft bone. A central zone of the endochondral interface is observed at the center of fusion mass, uniting the lower and upper halves of fusion. Pluripotent cells in this central zone differentiate into cartilaginous tissue with less vascularization.

During remodeling at weeks 6-10, a peripheral cortical rim forms around fusion. Bone marrow activity increases, forming secondary spongiosa. The cortical rim thickens. The trabecular process extends to the center of fusion. Remodeling typically completes one year after device implantation.

Pseudarthrosis (nonunion) was a leading cause of pain postoperatively and accounted for 45%-56% of revisions. Boney fusion directly correlates to successful clinical outcomes. Patients with pseudarthrosis were asymptomatic in about 30% of cases. Younger age has a significantly increased symptomatic pseudarthrosis rate (43.8 years vs. 52.1 years, p<0.01).

In certain embodiments, bone marrow aspirate (BMA) with allograft substitutes autogenous bone graft in single-level revision posterolateral lumbar fusion (PLF). In certain embodiments, bone marrow aspirate with allograft is more cost-effective than recombinant human bone morphogenetic protein-2 (rhBMP). In certain embodiments, bone marrow-derived cell-enriched allografts compare to autografts in bone grafting and spinal fusion procedures. In certain embodiments, BMA increases the regenerative potential of corticocancellous allogeneic bone grafts. When treating unicameral bone cysts, healing rates were high (98.7%) for bone marrow with demineralized bone matrix injection.

When introducing elements of the present disclosure or the embodiments(s) thereof, the articles “a,” “an,” “the,” and “said” are intended to mean that there are one or more of the elements. The terms “comprising,” “including,” and “having” are intended to be inclusive and mean that there may be additional elements other than the listed elements.

Having described the disclosure in detail, it will be apparent that modifications and variations are possible without departing from the scope of the disclosure defined in the appended claims.

Although the disclosure described herein is susceptible to various modifications and alternative iterations, specific embodiments thereof have been described in greater detail above. It should be understood, however, that the detailed description of the composition is not intended to limit the disclosure to the specific embodiments disclosed. Rather, it should be understood that the disclosure is intended to cover all modifications, equivalents, and alternatives falling within the spirit and scope of the disclosure as defined by the claim language.

### EXAMPLES

The following examples are included to demonstrate certain embodiments of the disclosure. It should be appreciated by those of skill in the art that the techniques disclosed in the examples represent techniques discovered by the inventors to function well in the practice of the disclosure. Those of skill in the art should, however, in light of the present disclosure, appreciate that many changes can be made in the specific embodiments disclosed and still obtain a like or similar result without departing from the spirit and scope of the disclosure. Therefore, all matter is interpreted as illustrative and not in a limiting sense.

TABLE 1
Reference numerals

| | |
|---|---|
| 100 | device |
| 200 | pedicle screw |
| 210 | proximal end |
| 220 | distal tip |
| 230 | thread |
| 231 | leading edge |
| 232 | trailing edge |
| 235 | leading surface |
| 236 | trailing surface |
| 240 | shaft |
| 250 | openings |
| 251 | first opening |
| 252 | second opening |
| 260 | canula/core |
| 270 | cutting member |
| 271 | cutting edge |
| 280 | scaffold |
| 300 | pedicle screw |
| 400 | pedicle screw |
| 500 | pedicle screw |
| 510 | cap |
| 511 | cap top |
| 512 | cap bottom |
| 513 | cap thread |
| 514 | cap body |
| 520 | saddle |
| 521 | saddle top |
| 522 | saddle bottom |
| 523 | saddle groove |
| 524 | saddle body |
| 530 | thread |
| 540 | shaft |
| 590 | tulip |
| 591 | top opening |
| 592 | bottom opening |
| 593 | tulip groove |
| 594 | tulip body |
| 595 | pin |
| 596 | side opening |

### Example 1—Pedicle Screw

Referring to FIGS. 2-4, the pedicle screw 200 was 3D-printed in titanium. The pedicle screw 200 comprised a thread 230 disposed around a shaft 240 that extends between the proximal end 210 and distal tip 220. The thread 230 comprised an external thread form with a leading edge 231 having a leading surface 235 and a trailing edge 232 having a trailing surface 236. The leading surface 235 defined a first opening 251. The trailing surface 236 defined a second opening 252. The first and second openings 251,252 were axially aligned. The pedicle screw 200 had a core 260 extending through the center of the pedicle screw 200 from the proximal end 210 to the distal tip 220. The distal tip 220 comprised two cutting members 270, each having a cutting edge 271

Referring to FIGS. 5-7, an embodiment of a pedicle screw 300 was 3D-printed in titanium with a scaffold 280 disclosed herein. The pedicle screw 300 comprised a thread 230 disposed around a shaft 240 that extends between the proximal end 210 and distal tip 220. The shaft 240 comprises two regions of scaffold 280 exposed to the outer surface of the pedicle screw 300. The thread 230 comprised an external thread form with a leading edge 231 having a leading surface 235 and a trailing edge 232 having a trailing surface 236. The leading surface 235 defined a first opening 251. The trailing surface 236 defined a second opening 252. The first and second openings 251,252 were axially aligned. The pedicle screw 300 had a core 260 filled with scaffold 280 extending through the center of the pedicle screw 300 from the proximal end 210 to the distal tip 220. The distal tip 220 comprised three cutting members 270, each having a cutting edge 271.

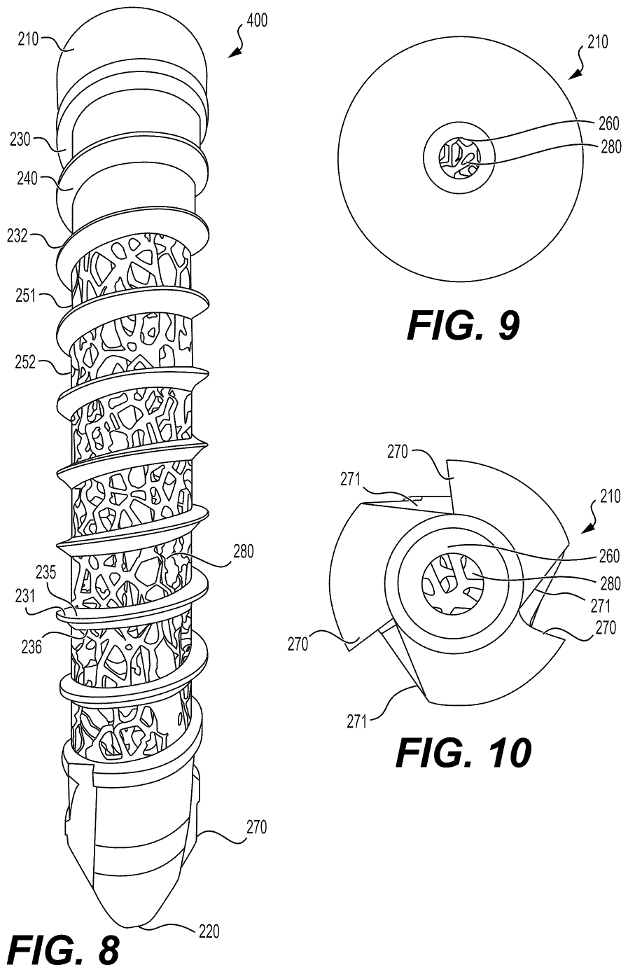

Referring to FIGS. 8-10, another embodiment of a pedicle screw 400 was 3D-printed in titanium with scaffold 280 disclosed herein. The pedicle screw 400 comprised a thread 230 disposed around a shaft 240 that extends between the proximal end 210 and distal tip 220. The shaft 240 comprises regions of scaffold 280 exposed to the outer surface of the pedicle screw 400 between the middle seven turns of the thread 230. The thread 230 comprised an external thread form with a leading edge 231 having a leading surface 235 and a trailing edge 232 having a trailing surface 236. The leading surface 235 defined a first opening 251. The trailing surface 236 defined a second opening 252. The first and second openings 251,252 were axially aligned. The pedicle screw 400 had a core 260 filled with scaffold 280 extending through the center of the pedicle screw 400 from the proximal end 210 to the distal tip 220. The distal tip 220 comprised three cutting members 270, each having a cutting edge 271.

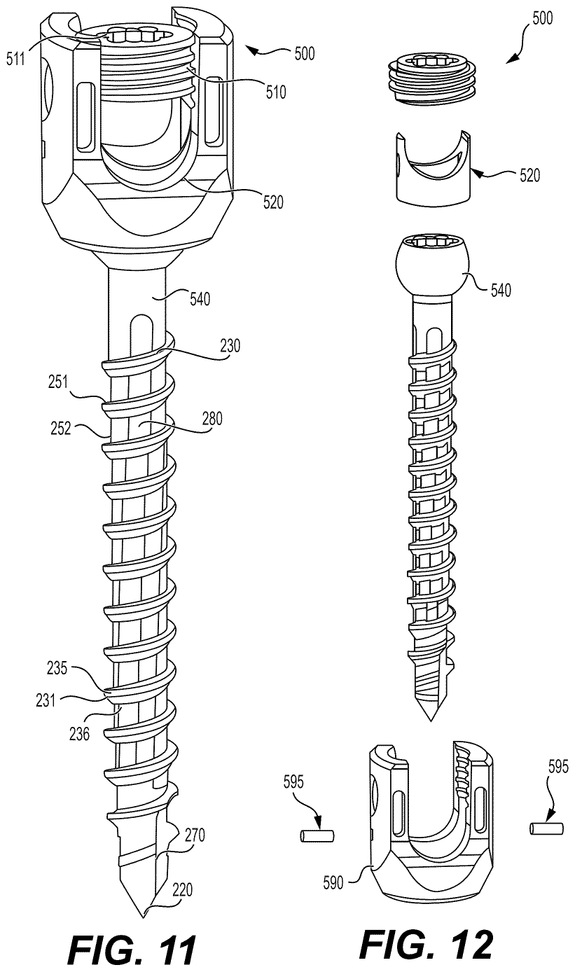

Referring to FIGS. 11-16, another embodiment of a pedicle screw 500 was 3D-printed in metal with scaffold 280 disclosed herein. The pedicle screw 500 comprises a cap 510, saddle 520, and shaft 540, and, when present, a tulip 590 and a pair of pins 595. The cap 510 is configured to couple to the saddle 520 via cap threads 513 operatively disposed in saddle grooves 523. The saddle 520 is configured to couple to the shaft 540. In embodiments of the pedicle screw having a tulip, the distal tip 220 of the shaft 540 can be disposed through the bottom opening 592 of the tulip 590 and held into place with pins 595 disposed through the side openings 596 of the tulip 590.

The pedicle screw 500 comprises a thread 230 disposed around a shaft 240 that extends between the proximal end 210 and distal tip 220. The shaft 240 comprises regions of scaffold 280 exposed to the outer surface of the pedicle screw 500 between the middle thirteen turns of the thread 230. The thread 230 comprises an external thread form with a leading edge having a leading surface and a trailing edge having a trailing surface. The pedicle screw 500 had a core filled with scaffold 280 extending through the center of the pedicle screw 500 from the proximal end 210 to the distal tip 220. The distal tip 220 comprised cutting members 270.

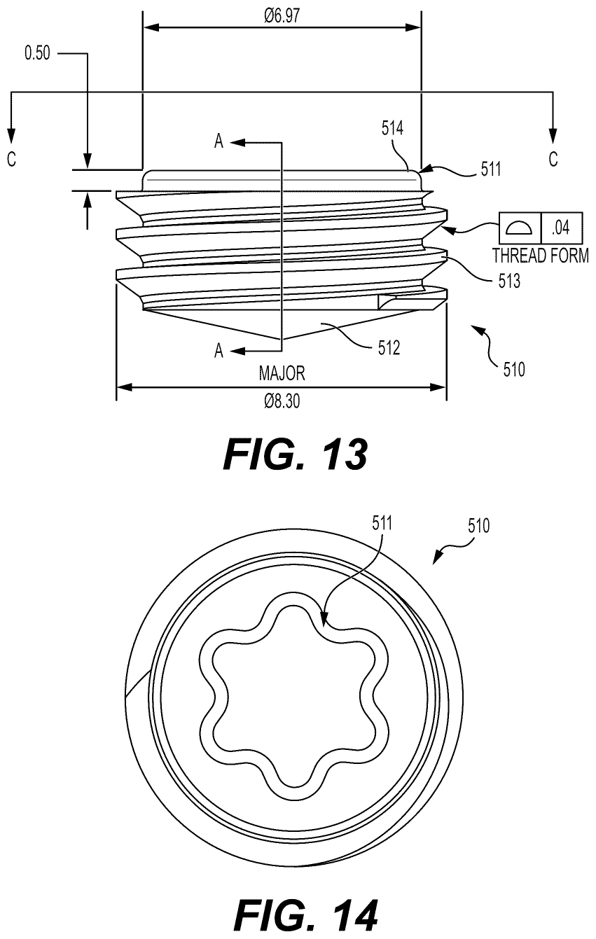

Referring to FIGS. 13 and 14, the cap 510 comprises a cap body 514 having a cap thread 513 disposed in a spiral around the outer surface of the cap body 514 between the cap top 511 and the cap bottom 512.

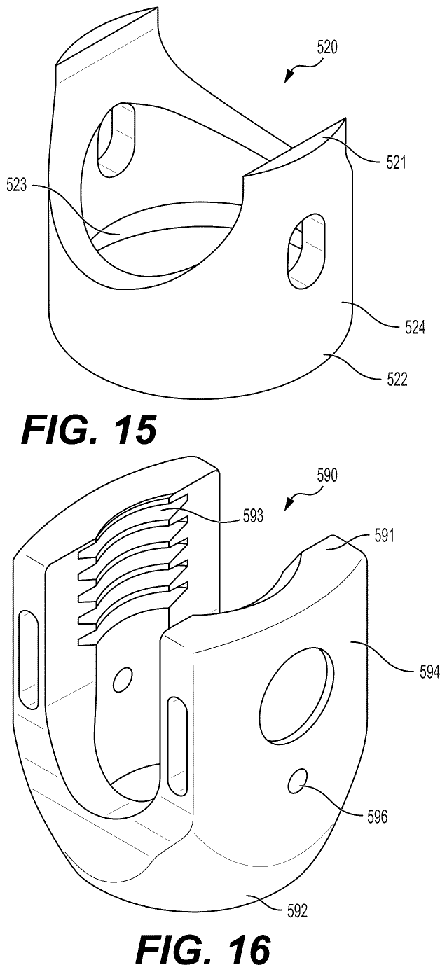

Referring to FIG. 15, the saddle 520 comprises a saddle body 524 having a saddle top 521, a saddle bottom 522, and at least one saddle groove 523 disposed on the interior surface of the saddle body 524. The at least one saddle groove 523 is configured to receive and operatively couple the cap threads 513. Likewise, the saddle bottom 522 is configured to receive and operatively couple the proximal end 210 of the shaft 540.

Referring to FIG. 16, when present, the tulip 590 comprises a tulip body 594 having a top opening 591, bottom opening 592, at least one pair of tulip grooves 593 disposed on the interior surface of the tulip body 594, a pair of side openings 596 between the at least one pair of tulip groove 593 and the bottom opening 592. The at least one pair of tulip grooves 593 are configured to receive and operatively couple the threads 230 of the shaft 540 when the distal tip 220 of the shaft 540 passes through the bottom opening 592 of the tulip 590. After the threads 230 have engaged the at least one pair of tulip grooves 593, a pair of pins 595 can be operatively coupled to the tulip 590 through the pair of side openings 596.

When present, the pores of the scaffold 280 promoted boney in-growth through the screw. Other materials fabricating the pedicle screw included pre-packed demineralized bone matrix (DBM), pre-packed synthetic DBM, unpacked DBM, and magnesium-infused titanium. Built-in channels captured autograft during insertion. The screw had double ball angulation and a low profile. The screw comprised a locking cap with reverse-angle threads. The screw can be cannulated or non-cannulated.

The screws were between 35 mm and 65 mm long and between 4.5 mm and 8.5 mm in diameter. The rod acceptance was 5.5 mm.

Built-in channels for autograft collection enhance the structural integrity of the implantation. These superiorly resisted bone mineral density loss and reduced micromotion. The randomized porosity pattern of the scaffold 280 was typical of native trabecular bone. In addition, built-in struts provided structural integrity. The pedicle screws 200,300,400,500 were manufactured in cobalt chrome, titanium, and magnesium-infused titanium.

The device was tested in cobalt chrome and met American Society for Testing and Materials (ASTM) standards 543, 1798, and 1717.

ASTM standard 543 evaluates plastic materials for resistance to chemical reagents, including cast, hot-molded, cold-molded, laminated resinous products, and sheet materials. Three procedures are presented, two under practice A (Immersion Test) and one under practice B (Mechanical Stress and Reagent Exposure under Standardized Conditions of Applied Strain). These practices report changes in weight, dimensions, appearance, color, strength, and other mechanical properties. Standard reagents are specified to establish results on a comparable basis without precluding other chemical reagents pertinent to specific chemical resistance requirements. Provisions are made for various exposure times, stress conditions, and exposure to reagents at elevated temperatures. The type of conditioning (immersion or wet patch/wipe method) depends upon the material's end-use.

ASTM standard 1798 covers the measurement of uniaxial static, fatigue strength, and resistance to loosening the component interconnection mechanisms of spinal arthrodesis implants. This test method provides a means of mechanically characterizing different designs of spinal implant interconnections. The various components and interconnections may be combined for static and fatigue testing of the spinal implant construct. This test method does not address the analysis of spinal implant constructs or subconstructs or define levels of performance of spinal implants.

ASTM standard 1717 covers the materials and methods for the static and fatigue testing of spinal implant assemblies in a vertebrectomy model. The test materials for combinations of spinal implant components can be specific, depending on the spinal location and intended application method to the spine. These test methods provide a basis for the mechanical comparison among past, present, and future spinal implant assemblies. They allow the comparison of spinal implant constructs with different intended spinal locations and application methods to the spine. These test methods are not intended to define levels of performance. Instead, these test methods set out guidelines for load types and methods of applying loads, measuring displacements, determining the yield load, and evaluating the stiffness and strength of the spinal implant assembly. Methods for three static load types and one fatigue test are defined for the comparative evaluation of spinal implant assemblies.

In certain embodiments, the pedicle screw 200,300,400,500 is individually packaged in a double Tyvek™ peel tray.

In certain embodiments, the pedicle screw 200,300,400,500 is injected or sprayed with a material, such as BMA concentrate, calcium phosphate, biologic, and/or antibiotics. The filled or coated screw rested for 10-15 minutes before insertion for the material to absorb.

### Example 3—Bone Harvesting Screws

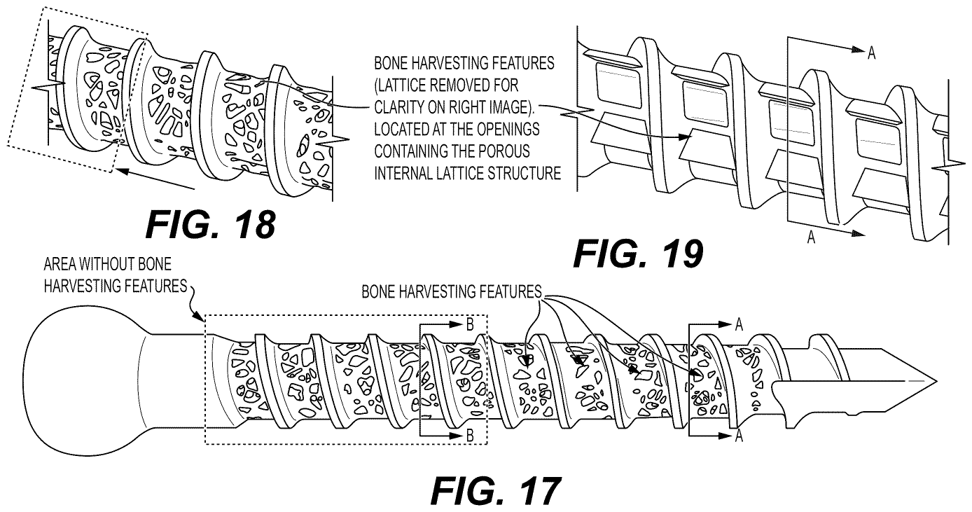

Referring to FIG. 17, bone harvesting features (scoops) are located on the minor diameter of the screw. These pull bone and cells into the lattice structure as the screw is inserted.

In certain embodiments, the bone harvesting features are located along the entire length of the screw.

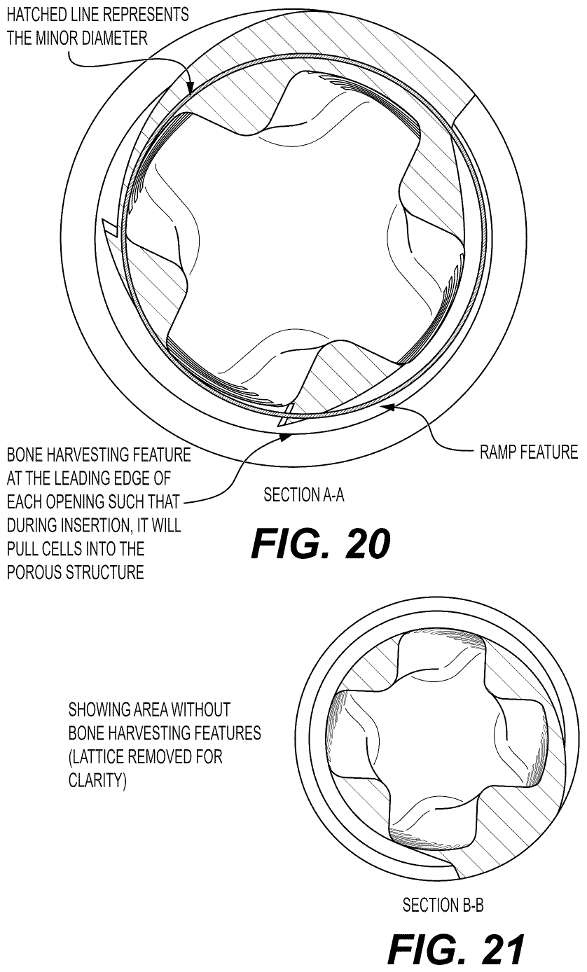

In certain embodiments, the bone harvesting features are located along the distal portion of the screw only (FIG. 19). Cross-section A-A shows the bone harvesting feature at the leading edge of each opening, such that during insertion, the bone harvesting feature pulls cells into the porous (lattice) structure of the screw (FIG. 20.) In this embodiment, the proximal portion does not have these features (FIG. 18), permitting a tight fit in cortical bone situated in the proximal end of the screw. Cross-section B-B shows an area without bone harvesting features (FIG. 21).

In certain embodiments, scoop features ramp from the minor diameter to a diameter slightly greater than the minor diameter on the surface between the porous structure. In such embodiments, the scoop features are biased in the direction of insertion.

In certain embodiments, the scoop features are angled or have a radius to assist with harvesting. In certain embodiments, the leading edge is angled.

In certain embodiments, the screw comprises a thickened head-neck junction below the aperture, for example, to reduce the likelihood of rod breakage during screw installation.

### Example 3—Diamond Lattice Structure

Bone screws were 3D-printed and tested with the diamond lattice structure. In certain embodiments, scoop features are located along a helical pattern, for example, corresponding to the helical pattern of the openings into the internal lattice structure.

The design was based on a triply periodic minimal surface (TPMS) is a minimal surface in R³ that is invariant under a rank-3 lattice of translations. These surfaces have the symmetries of a crystallographic group. Numerous examples are known with cubic, tetragonal, rhombohedral, and orthorhombic symmetries.

Specifically, a Schwartz Diamond TPMS was used for the lattice, formed from symmetry arguments, remapped from Cartesian coordinates to spherical polar coordinates about the central axis of the screw shaft, sheared to form a helical wrap, thickened, subtracted, and as intersected with the 3D geometry space for the scaffold.

The surfaces were generated using symmetry arguments: Given a solution to Plateau's problem for a polygon, reflections of the surface across the boundary lines also produce valid minimal surfaces that can be continuously joined to the original solution. If a minimal surface meets a plane at right angles, then the mirror image in the plane can also be joined to the surface. Hence, given a suitable initial polygon inscribed in a unit cell, periodic surfaces can be constructed.

The equation 1 approximates the TPMS for these bone screws:

$$cos ( x ) cos ( y ) cos ( z ) - sin ( x ) sin ( y ) sin ( z ) = 0 \qquad ( 1 )$$

which is the specific base equation for this embodiment of the bone screw. The x, y, and z variables define the periodicity (i.e., pattern) in X/Y/Z, similar to how cubic lattices are defined. This surface is called a “diamond” because it has two intertwined congruent labyrinths, each having the shape of an inflated tubular version of the diamond bond structure. For the sake of discussion, a regularly repeating cell has been assumed, although TPMS geometry is influenced topologically to be pseudo-random. An exact expression exists in terms of elliptic integrals based on the Weierstrass-Ennepar parameterization.

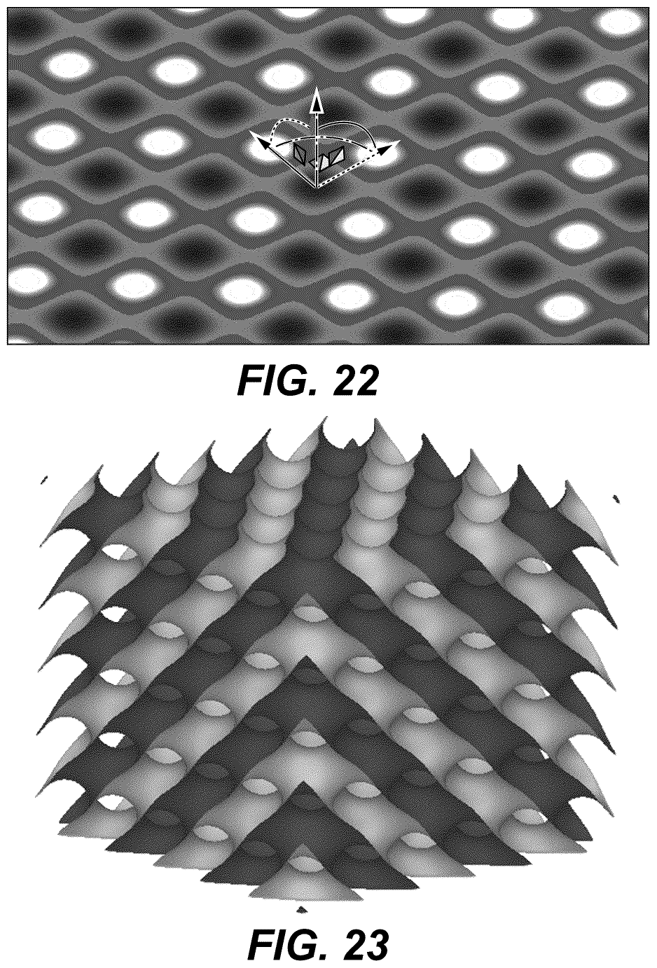

As an equation, it defines the Schwarz D surface through infinite real space Such a surface splits real space into two identical volumes—Positive space passes into negative space defines the iso- or mid-surface. FIG. 22 illustrates the 2-dimensional slice through the Schwarz Diamond mathematical field, where teal space is “positive” and purple space is “negative.” FIG. 23 illustrates a 3-dimensional cross-section of the non-thickened surface from FIG. 22, where light gray represents the positive side of the surface and dark gray the negative side, as defined by surface normal vectors. In certain embodiments, the cubic repeating pattern is 1.8 mm in X/Y/Z.

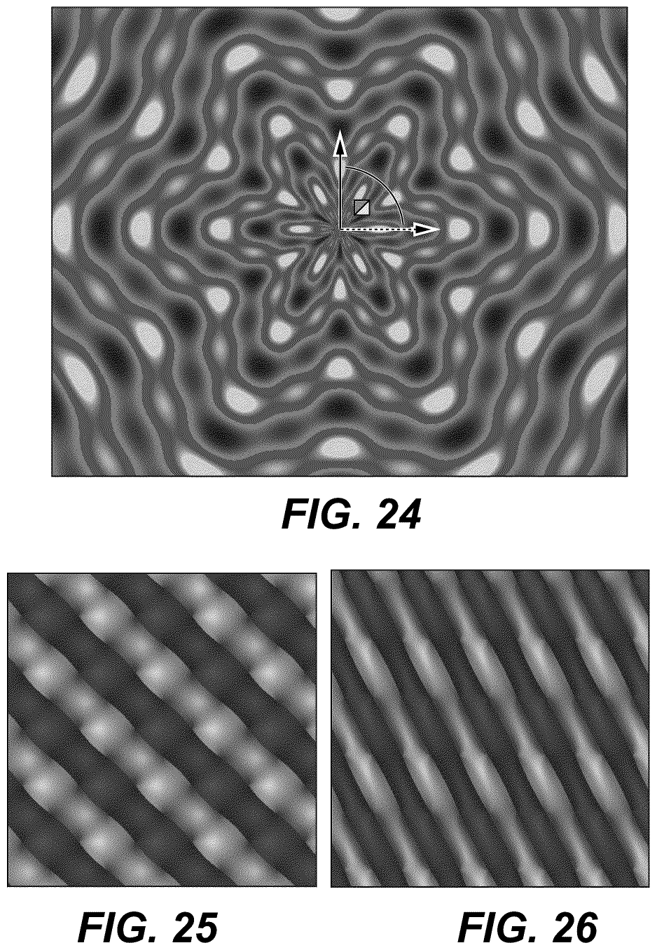

After creating the Schwarz equation, it was remapped helically (i.e., twisted) to create the base of the final shape. To do this, the equation was mapped from Cartesian space to polar space using conventional methods. The periodicity was mapped cylindrically. That is, the number of “spokes” radially remained a multiple of the selected cell size. FIG. 24 shows the cylindrical remap after conversion to polar space. The remap was about the central axis of the screw shaft.

After remapped, the space was sheared to create the helical wrap, similar to how an inclined plane is wrapped around a cylinder to create a screw. To create the shear, the Schwarz D equation is remapped from X/Y/Z coordinate space by shearing one (or multiple of the coordinates): x→x, y→y, and z→z+x, where the Schwarz Diamond was sheared in the XZ plane. Such a shear operation maintained a continuous field.

After the field was sheared and remapped cylindrically, it was thickened using an absolute value operation, which converted the negative space of the equation into positive in three dimensions (FIG. 25). The helical wrap of the medical device as defined with a period three times the size of the cubic repeating pattern (5.4 mm), forming a single helix with a circumferential count of three for three radial spokes. After this, a subtract mathematical operation offset the central geometry to create a sheet-like structure, as seen in FIG. 26, wherein thin-wall geometry is represented by the thin pink wall radiating cylindrically. In certain embodiments, the walls are about 0.50 mm thick. Once the thin-walled lattice field was intersected with the 3D geometry space that defines where the lattice exists, to model for the scaffold was produced (FIG. 29).

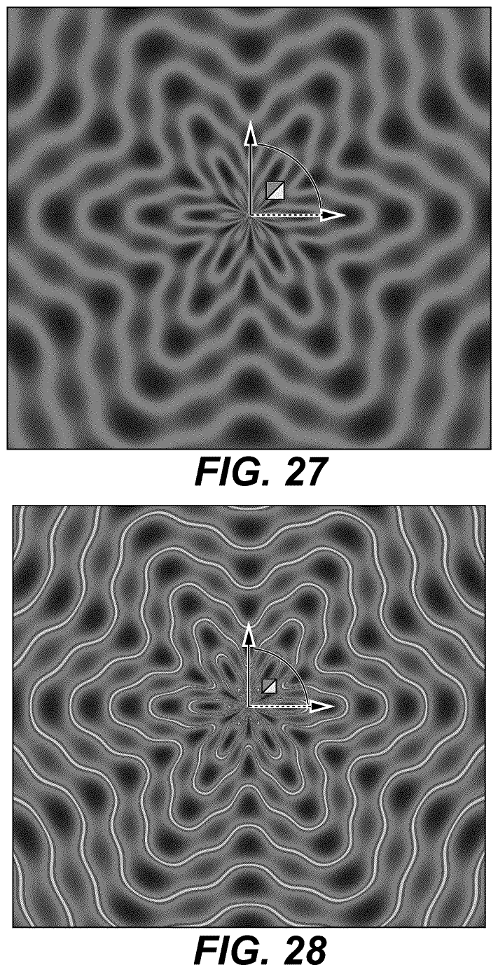

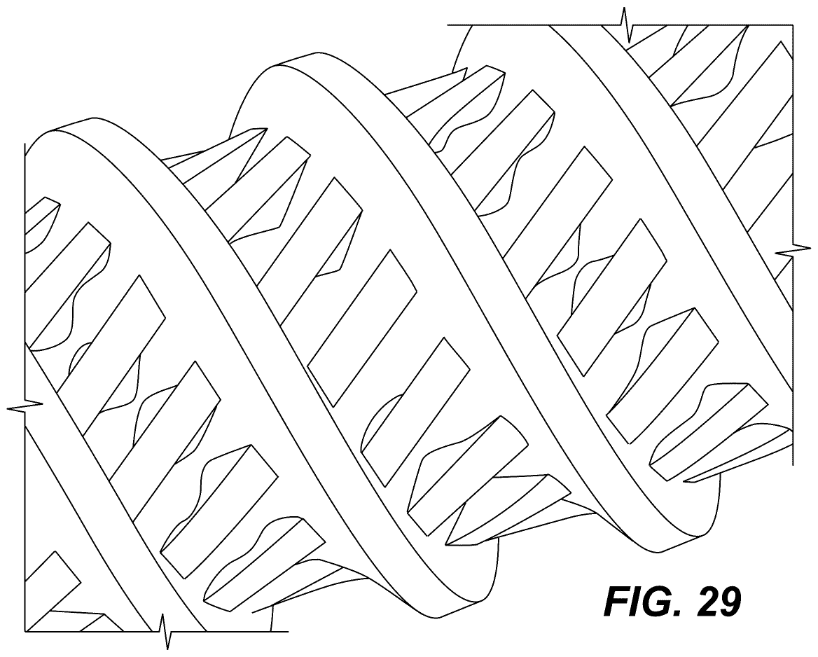

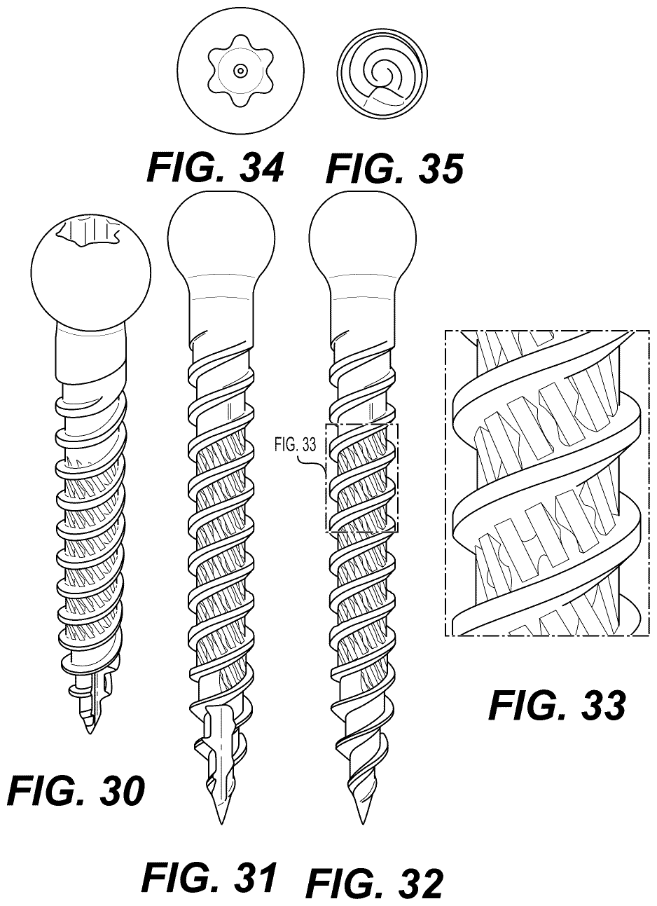

The diamond lattice described above was produced for two embodiments of the bone screw. FIG. 30 shows a perspective view of one embodiment of a porous pedicle screw presenting such a helical pattern in a diamond-structured lattice. FIG. 31 shows the back, FIG. 32 the front, FIG. 34 the top, and FIG. 35 the bottom of the porous pedicle screw. FIG. 33 shows a magnified inset of the front view of the porous pedicle screw.

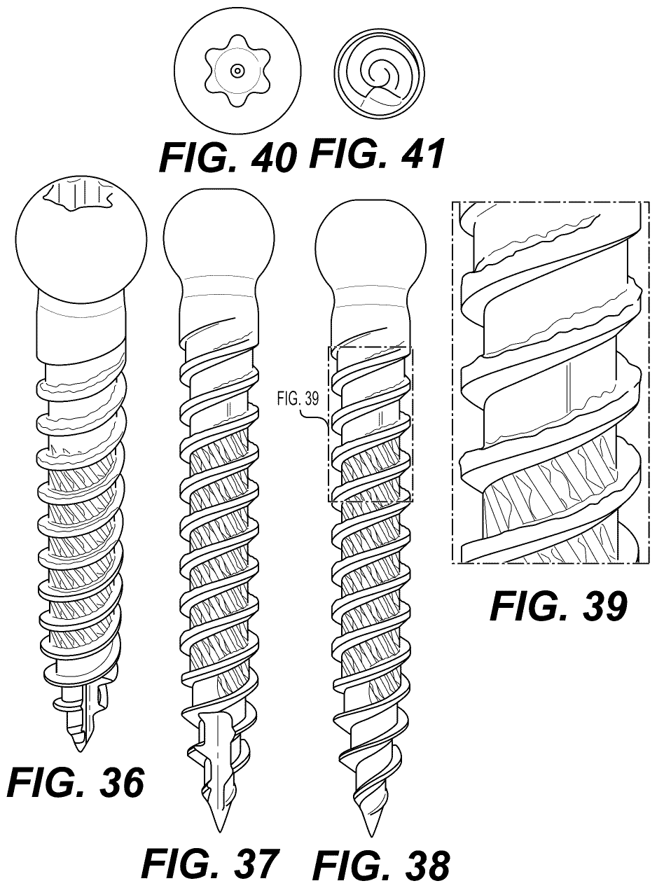

FIG. 36 shows a perspective view of another embodiment of a porous pedicle screw presenting a diamond-structured lattice comprising bone harvesting features at the distal tip and textured threads at the proximal end. The texture resembles the surface topography of the diamond lattice structure. FIG. 37 shows the back, FIG. 38 the front, FIG. 40 the top, and

FIG. 41f the bottom of the porous pedicle screw. FIG. 39 shows a magnified inset of the front view of the porous pedicle screw.

### Example 4—Sheep Study for Bone Integration and Pullout Strength

In-vivo assessments, ex-vivo assessments, and data from this six sheep study will determine how this treatment modality affects bone mineral density, polymorphonuclear cells (PMNs), lymphocytes, plasma cells, macrophages (Mφ), giant cells, necrosis, osteoblastic cells, signs of bone remodeling by osteoclasts, neovascularization, fibrosis, signs of implant degradation, and particulate debris.

The first specific aim is to determine whether the porous pedicle screw promotes bone integration and pullout strength compared to the gold-standard pedicle screw/rod constructs in a posterior lumbar interbody fusion sheep model. The topography of 3D printed porous patterns has higher adhesion of stem cells to titanium. In addition, mesenchymal and hematopoietic stem cells have therapeutic effects on bone. By combining these two modalities, superior results can be achieved in the disclosed porous pedicle screws concerning bone integration and pullout strength over current pedicle screws.

To this end, the bone mineral densities (BMD) of 84 vertebral bodies (L₁-L₆) will be measured from six sheep one week preoperatively and postoperatively at 24 and 36 weeks. Each subject will receive two separate lumbar interbody fusions (LIF) at the L₂-L₃ and L₄-L₅ joints. L₁ and L₆ will be naïve controls to compare changes with and without hardware.

TABLE 2
Animal subjects

| | | | | | CT/ | |
|---|---|---|---|---|---|---|
| Animal | | | L₁, | Imaging | Syngo- | Sacri- |
| ID | L₂-L₃ | L₄-L₅ | L₆ | Rads | Osteo | fice |
| 1 | Control | Treatment | Naïve | Post-op, | Sacrifice | 36 |
| | | | | Sacrifice | | weeks |
| 2 | Treatment | Treatment | Naïve | Post-op, | Sacrifice | 36 |
| | | | | Sacrifice | | weeks |
| 3 | Control | Control | Naïve | Post-op, | Sacrifice | 36 |
| | | | | Sacrifice | | weeks |
| 4 | Treatment | Treatment | Naïve | Post-op, | Sacrifice | 36 |
| | | | | Sacrifice | | weeks |
| 5 | Control | Control | Naïve | Post-op, | Sacrifice | 36 |
| | | | | Sacrifice | | weeks |
| 6 | Control | Treatment | Naïve | Post-op, | Sacrifice | 36 |
| | | | | Sacrifice | | weeks |

In each subject, a titanium interbody cage and bone void filler packed into the interbody cage will be placed between the L₂-L₃ and L₄-L₅ segments. Then, 4.5, 5.5, or 6.5-mm diameter and 45+10 mm screws will be inserted into the right and left pedicles within the L₂, L₃, L₄, and L₅ vertebral bodies. This configuration represents traditional fusion devices and surgical techniques. Before insertion, the porous pedicle screws (treatment) will be sprayed with autologous stem cell concentrate along the length of the porous portion screw.

In-life lumbar spine radiographs will be performed on all animals immediately post-op (PO) and at sacrifice. Animals will be visually assessed at least once daily throughout the study. Abnormalities, such as signs of infection at the surgical site, will be recorded. A total of 6 animals will be sacrificed 36 weeks after surgery.

Following euthanasia, lumbar spine sections (L₁-L₅) will be freshly dissected to a single functional spinal unit (FSU) (i.e., L₄-L₅) for post-sacrifice assessments. High-resolution biplanar digital radiographs and photos will be taken at sacrifice following fine dissection in the sagittal and coronal planes. Non-destructive range of motion (ROM) biomechanics will be measured on all samples, including ROM biomechanics under pure moment loading in flexion-extension, lateral bending, and axial rotation to 6.0 N-m, yielding range of motion (Degrees), construct stiffness (Deg./N-m), and neutral zone (Deg.).

Destructive pedicle screw pullout will be tested. Quasi-static ramp to failure testing will yield construct stiffness (N/mm), yield force (N), ultimate failure force (N), and mode of failure (MOD) observed visually. Destructive pedicle screw torque-out will be tested for N=1 of 4 screws from each, and quasi-static torque counterclockwise to loosen the screw will yield ultimate torque (Nm).

Other tests will include micro-computed tomography (MicroCT) of each FSU and associated pedicle screws, quantitative assessment of the posterior lumbar fusion (PLF) region (bone volume and bone density), qualitative assessment of bone ingrowth around pedicle screws, pedicle screw histology, organ histology, and static histomorphometry of screw regions of interest (ROIs), including the percentage of the bone area within ROI, percentage of fibrous tissue within ROI, percentage of void space with ROI, percentage of the screw within ROI, and percentage of bone on-growth to the device.

Slides will be delivered to a certified pathologist for histopathology analysis. The pathologist will be initially blinded to the treatment parameters of each site. Then, when applicable, the sections will be analyzed and graded per cell type and responses following the grading scheme in Table 3. After scoring all the slides for data post-processing, the pathologist will be unblinded so they can compare data to the control samples.

TABLE 3
Scoring system for histological evaluation
of bone sections for cell type and response

| | Score | | | | |
|---|---|---|---|---|---|
| Cell type/response | 0 | 1 | 2 | 3 | 4 |
| Polymorphonuclear | None | Rare, | 5-10/HPF | Heavy | Packed |
| cells (PMNs) | | 1-5/HPF* | | infiltrate | |
| Lymphocytes | None | Rare, | 5-10/HPF | Heavy | Packed |
| | | 1-5/HPF | | infiltrate | |
| Plasma cells | None | Rare, | 5-10/HPF | Heavy | Packed |
| | | 1-5/HPF | | infiltrate | |
| Macrophages (Mφ) | None | Rare, | 5-10/HPF | Heavy | Packed |
| | | 1-5/HPF | | infiltrate | |
| Giant cells | None | Rare, | 3-5/HPF | Heavy | Sheets |
| | | 1-2/HPF | | infiltrate | |
| Necrosis | None | Minimal | Mild | Moderate | Severe |
| Osteoblastic cells⁺ | None | Minimal | Mild | Moderate | Severe⁺ |
| Signs of bone | None | Minimal | Mild | Moderate | Severe^{#} |
| remodeling by | | | | | |
| osteoclasts^{#} | | | | | |
| Neovascu- | None | Minimal | Mild | Moderate | Severe |
| larization ^{&} | | | | | |
| Fibrosis | None | Minimal | Mild | Moderate | Severe |
| Signs of implant | None | Minimal | Mild | Moderate | Severe |
| degradation | | | | | |
| Particulate debris | None | Minimal | Mild | Moderate | Severe |

*HPF - per high-powered field
⁺Osteoblastic cells - severe suggests numerous OB Cells
^{#}Signs of bone remodeling by osteoclasts - severe suggests abundant bone remodeling present
^{&} Neovascularization - severe suggests numerous neovascular vessels
Reference: ISO 10993-6 Annex E (Biological evaluation of medical devices - Part 6: Tests for local effects after implantation)

The histopathology report will include, but will not be limited to, a summary of methods and materials, tabulated and qualitative data through the last time point and conclusions, low-power images, and representative photomicrographs to illustrate the findings. An unpaired t-test with an alpha (α) value of 0.05 will be performed to determine statistical significance for biomechanical and histomorphometric outcome parameters. Then, the data will be compared with similar retrospective studies.

This study's second specific aim is to show that injecting and spraying autologous concentrated stem cells within and around pedicle screws is safe. Porous 3D-printed titanium interbody cages are commonly impregnated intraoperatively with autologous stem cells. They have been proven safe and are the gold standard to aid fusion between the vertebrae after removing the disc. This study aims to prove the same can be performed within the vertebral bone in sheep to provide confidence of safety for a human clinical trial.

After sacrifice, histology will be compared with prior studies to determine the differences and similarities of polymorphonuclear cells (PMNs), lymphocytes, plasma cells, macrophages (MP), giant cells, necrosis, osteoblastic cells+, signs of bone remodeling by osteoclasts, neovascularization, fibrosis, signs of implant degradation, and particulate debris. Histology reports will also be compared and contrasted between the control, naïve, and treatment sites. An unpaired t-test with an alpha (α) value of 0.05 will be performed to determine statistical significance for biomechanical and histomorphometric outcome parameters. Injecting autologous stem cells within and around the porous pedicle screws is expected to be safe compared to the control screws, naïve screws, and prior studies.

This study's third specific aim is to show that porous pedicle screws have a topography and porous pattern for promoting stem cell adhesion. Human mesenchymal stem cells have the strongest adhesive affinity for titanium surfaces with porosities between 50% and 70%, a more robust and dense internal cellular migration pattern, and high cell viability. Therefore, the porous pattern and topography of porous pedicle screws should have a similar adhesion to stem cells.

After the sheep have been sacrificed, the screws will be removed from the vertebrae and studied for stem cell adhesion. Cell viability on the implant surface will be performed with a LIVE/DEAD assay. A conditioned media assay will be used to study bone morphogenic protein 2 (BMP2) expression levels, vascular endothelial growth factor (VEGF), osteocalcin, osteoprotegerin expression, DNA, and alkaline phosphatase activity.

The correlation between cell adhesion with 3D printed titanium patterns and porous pedicle screws will be shown. Porous pedicle screws demonstrate better stem cell adhesion than the control and naïve subjects, as well as similar adhesion rates to prior studies.

### Example 5—Sheep Study for Infection

Another six-animal study will focus on testing the feasibility of injecting calcium sulfate with antibiotic mixtures as a means of reducing rates of infection following spinal fusions. The main objectives of this project are to confirm whether (1) the tested pedicle screw aids superior bone integration and pullout strength compared to the gold-standard pedicle screw/rod constructs in a posterior lumbar interbody fusion sheep model; (2) injecting calcium sulfate with antibiotic mixtures can reduce the rate of infection following spinal fusions; and (3) the tested pedicle screws have topography and porous pattern for supporting injection of the above goals.

For the first aim, the rationale is that, if a patient has an infected bone, surgeons can protect the hardware by injecting an antibiotic mixture through the device. Through the proposed animal study, we will confirm that, in an infected and contained area (e.g., vertebral bone), the pedicle screw will (1) protect surgical hardware (i.e., confirm infection has not spread into hardware) compared to controls and (2) reduce infection in the bone.

An ovine model was chosen because sheep have the spinal column most similar to the human spine. Sheep vertebrae are large enough to accommodate a pedicle screw disclosed herein. Smaller animals are not viable because the screws are too large for their bones.

This sample size was chosen to realistically assess feasibility and achieve proof-of-concept within a Phase I scope and timeline. In alignment with program objectives, Phase I results will be interpreted as preliminary and tentative conclusions will be used to inform an anticipated Phase II where we can propose a large, controlled, well-powered animal study that evaluates efficacy endpoints in a scientifically rigorous manner.

The experimental design and methods will be substantially the same as the sheep study above in Example 4, including Tables 2 and 3.

All references, patents, or applications, US or foreign, cited in the application are because of this incorporated by reference as if written herein in their entireties. Where any inconsistencies arise, the material disclosed herein controls.

From the preceding description, one skilled in the art can easily ascertain the essential characteristics of this invention and, without departing from the spirit and scope thereof, can make various changes and modifications of the invention to adapt it to various usages and conditions.

1. A medical device comprising:
a body;
a scaffold in the body; and
disposed on the body, a means for focusing bone growth throughout the body to minimize shear stresses on a distal tip and spread micromotion throughout the medical device to encourage bony ingrowth.

2. The medical device of claim 1, wherein the means for focusing bone growth comprises at least one trephine to harvest bone internally within the medical device.

3. The medical device of claim 1, configured in arcuate cross-section patterns varied from a proximal end to a distal tip.

4. The medical device of claim 3, configured for placement into an internal cavity of a vertebral body such that, upon coaxial rotation of the medical device, an autograft is harvested within the scaffold.

5. The medical device of claim 1, having a proximal end and further comprising threads having a concave profile.

6. The medical device of claim 1, wherein the scaffold comprises a triply periodic minimal surface (TPMS) with a cubic repeating pattern defining walls within the scaffold.

7. The medical device of claim 6, wherein the TPMS is a Schwartz Diamond helically wrapped around a central axis of the medical devices and into 3D geometry space for the scaffold.

8. The medical device of claim 7, wherein the TPMS is helically wrapped into a single helix.

9. The medical device of claim 8, having three radial spokes per turn of the helix.

10. The medical device of claim 6, wherein the cubic repeating pattern is about 1.8 mm in X/Y/Z dimensions.

11. The medical device of claim 7, wherein the helical wrap of the medical device is defined with a period three times the size of the cubic repeating pattern.

12. The medical device of claim 6, wherein the walls are about 0.5 mm thick.

13. The medical device of claim 5, wherein the threads near the proximal end are textured with a topography resembling the topography of the scaffold.

14. The medical device of claim 1, configured to house one or more biologic agents.

15. The medical device of claim 1, further comprising at least one autologous product sprayed on or injected through the medical device.

16. (canceled)

17. The medical device of claim 1, which is a bone screw.

18. The medical device of claim 17, having reduced one or more of screw loosening, screw backout, rod breakage, and lowered bone mineral density.

19. The medical device of claim 17, comprising a fluted tip.

20. (canceled)

21. A method of treating a bone fracture in a patient in need thereof, comprising: implanting a medical device into a bone of the patient,
the medical device comprising:
a body;
a scaffold in the body; and
disposed on the body, a means for focusing bone growth throughout the body to minimize shear stresses on a distal tip and spread micromotion throughout the medical device to encourage bony ingrowth.

22. A pedicle screw comprising:
a body with a proximal end;
a scaffold in the body;
threads disposed on the body; and
disposed on the body, a means for focusing bone growth throughout the body to minimize shear stresses on a distal tip and spread micromotion throughout the pedicle screw to encourage bony ingrowth;
wherein the threads have a concave profile and the threads near the proximal end are textured with a topography resembling the topography of the scaffold.
